(19) United States
(12) Patent Application Publication
(10) Pub. No.: US 2026/0234712 A1
(43) Pub. Date: Aug. 13, 2026

(54) DEVICES, SYSTEMS, AND RELATED METHODS FOR ASSESSING LOCUS-SPECIFIC MOLECULAR HETEROGENEITY OF DNA METHYLATION

(71) Applicant: THE JOHNS HOPKINS UNIVERSITY, Baltimore, MD (US)

(72) Inventors: Thomas Russell PISANIC, II, Baltimore, MD (US); Christine O'KEEFE, White Marsh, MD (US); Tza-Huei Jeff WANG, Germantown, MD (US)

(21) Appl. No.: 19/474,921

(22) PCT Filed: Apr. 12, 2024
(86) PCT No.: PCT/US2024/024238

§ 371 Date: Oct. 13, 2025

Related U.S. Application Data
(60) Provisional application No. 63/496,108, filed on Apr. 14, 2023.

(51) Int. Cl. C12Q 1/686; C12Q 1/48; C12Q 1/6806; C12Q 1/6813; C12Q 1/6886

(52) U.S. Cl. CPC C12Q 1/686; C12Q 1/48; C12Q 1/6806; C12Q 1/6813; C12Q 1/6886; C12Y 207/07; C12Q 2600/118; C12Q 2600/154; C12Q 2600/16

(57) ABSTRACT

Provided herein are methods of determining methylation densities of target nucleic acids in a sample. In some aspects, the methods include amplifying target nucleic acids in multiplex reaction mixtures that comprise sets of methylation-agnostic ratiometrically-encoded nucleic acid probes to produce detected target nucleic acid loci. A given set of methylation-agnostic ratiometrically-encoded nucleic acid probes in a given reaction mixture comprises a sufficient number of sequence permutations to detect substantially all epialleles present at a given target nucleic acid locus and comprises a predetermined ratio of differential labeling that produces a detectable signal that is sufficient to distinguish the detected epialleles present at the given target nucleic acid locus from epialleles detected at other target nucleic acid loci. The methods also include determining a methylation status of each epiallele detected at each detected target nucleic acid loci. Provided also herein are kits, devices, systems, and other methods.

### CROSS-REFERENCE TO RELATED APPLICATIONS

This application is the national stage entry of International Patent Application No. PCT/US2024/024238, filed on Apr. 12, 2024, and published as WO 2024/216009 A9 on Oct. 17, 2024, which claims the benefit of U.S. Provisional Patent Application Ser. No. 63/496,108, filed Apr. 14, 2023, which are hereby incorporated by reference herein in their entireties.

### STATEMENT OF GOVERNMENT SUPPORT

This invention was made with government support under grants CA260628, CA272321, and CA086368 awarded by the National Institutes of Health. The government has certain rights in the invention.

### BACKGROUND

It is estimated that that over 1.8 million people in the U.S. will be diagnosed with cancer in the present year leading to 600,000 deaths and annual expenditures of over $137 billion dollars, 90% of which will be attributable to long-term and palliative care. Nonetheless, current calculations estimate that upwards of 25% of cancer-related deaths can likely be prevented by diagnosing cancer at earlier, more treatable stages, resulting in a net reduction in costs of over $25 billion dollars annually. One particularly attractive approach for cancer diagnostics is the use of circulating cell-free DNA (cfDNA) from so-called “liquid-biopsies” of patient-derived serum/plasma as these samples are often enriched in genetic material from tissues, including tumors, located throughout the body. However, tumor specific alterations, such as mutations and aberrant DNA methylation, are often only present at extraordinarily low copy numbers (<10 copies/ml) and fractional concentrations (<0.1%) within a large background of healthy-tissue DNA. This issue in particular has proven problematic for current technologies and has thus far precluded development of a cfDNA diagnostic method that is simple, low-cost and, most importantly, able to detect and identify cancers sufficiently early to improve patient outcomes.

Current cfDNA-based diagnostic methods generally rely on one of two means of detection: PCR or next generation sequencing (NGS). PCR-based techniques, including quantitative PCR (qPCR) and more recently, digital PCR (dPCR), are inexpensive and able to provide unparalleled, single-molecule sensitivity and precision, but their implementation with traditional fluorescence detection schemes has thus far precluded their ability to be multiplexed beyond 4-5 targets. On the other hand, NGS-based methods are able to provide targeted, highly parallelized analysis of many regions throughout the entire genome. However, even with the incorporation of so called unique molecular identifiers (UMIs), most NGS-based methods still generally struggle to detect variant fractions below 0.1%, resulting in only a limited ability to detect early-stage malignancies. While some recent, well-publicized approaches have sought to circumvent these fundamental limitations through multivariate (e,g., CancerSEEK) and/or extraordinary large biomarker panels (e.g., DELFI, cfMeDip-seq and the GRAIL project), these methods rely on complex proprietary algorithms that will need to be trained and validated with an inordinate number of samples (at least ten cases per predictor even according to the oft-referenced “one-in-ten” rule9) to prevent overfitting. Perhaps more importantly, these state-of-the-art sequencing and multivariate methods are invariably expensive, with current and anticipated costs to patients of up to several thousand dollars or more per test, likely limiting their ability to be widely and/or routinely implemented, particularly in disadvantaged populations. There thus remains a need for an approach that can not only reliably detect and identify common cancer types at early stages of disease, but is able to do so in a sufficiently cost-effective manner to enable the potential for routine (e.g., annual) cancer screenings for the population at large.

The concept of low-cost, early pan-cancer diagnostics is contingent upon identification of sensitive and specific biomarkers that can be detected at early stages of disease with inexpensive assay methods. Toward this end, biomarkers based on cancer-specific alterations in DNA methylation are a particularly attractive option. First, aberrant methylation is a notably early event in carcinogenesis, typically arising even prior to tumor development. Furthermore, DNA hypermethylation is inherently chemically and biologically stable and, unlike point mutations, often occurs in CpG islands that span hundreds to thousands of different regions throughout the cancer genome. This redundancy is particularly useful, as panels of methylation biomarkers can be utilized to improve overall clinical sensitivity and specificity, as well as to provide robustness against false positives stemming from clonal hematopoiesis that continue to plague mutation-based approaches. A further advantage of DNA methylation is that it is highly cell-type specific, which can be leveraged to identify tissues of origin of the tumor DNA found within liquid biopsies.

Yet despite these advantages, the detection of cancer-specific methylation at early stages of disease remains challenging. For example, while aberrant methylation is reportedly present in the vast majority of precursor lesions and early-stage tumors, hypermethylation typically arises progressively and in a stochastic manner leading to malignant cell populations with heterogeneous methylation profiles. This poses particular challenges for commonly employed techniques, such as methylation-specific PCR (MSP) and digital-MSP (dMSP) that are designed to detect only specific, predefined methylation patterns (typically densely-methylated), greatly limiting their ability to detect the rare, heterogeneously methylated epialleles that occur at early stages of cancer. On the other hand, sequencing-based approaches can analyze methylation patterns at thousands of loci, but simply lack the requisite sensitivity to achieve reliable detection at early stages of cancer.

Accordingly, it is apparent that there remains a need for an approach that provides a simple and inexpensive way to detect and quantify even partially-methylated epialleles with high sensitivity and high specificity.

### SUMMARY

This application discloses platforms and related aspects that provide a low-cost tool for evaluating and quantifying DNA methylation at single-molecule sensitivity on a copy-by-copy basis. In some embodiments, the methods of the present disclosure involve digitizing bisulfite- or enzymatically-converted DNA from nucleic acid samples into thousands of nanowells or droplets along with PCR reagents that include primer nucleic acids that are designed amplify regions of interest, regardless of methylation status, and nucleic acid probes that produce differential detectable signal signatures for each different target region of interest in a given sample. In these embodiments, PCR is then performed to detect target nucleic acids present in the sample and resulting amplicons undergo high-resolution melt (HRM). In some embodiments, these PCR and HRM steps are performed with the digitized reaction mixtures disposed in microfluidic devices. The characteristic HRM “melt temperature” (Tₘ) of amplicons derived from each respective template molecule (epiallele) is then typically assessed and used to directly infer the methylation density (fraction of CpG site methylated in the locus of interest) of the original template molecule. Using this approach, histograms of methylation statuses of all epialleles in the sample can be generated. The methods of the present disclosure provide very sensitive approaches to assessing locus-specific methylation. Some embodiments provide pan-cancer assays for low-cost screening and companion diagnostics of all major human cancers. These and other attributes will be apparent upon a complete review of the present disclosure, including the accompanying figures.

In one aspect, this disclosure provides a method of determining methylation densities of target nucleic acids in a sample. The method includes converting unmodified cytosine residues in nucleic acids from the sample to uracil residues to produce converted nucleic acids (i.e., converted [uracil] nucleic acids), and amplifying the target nucleic acids among the converted nucleic acids in multiplex reaction mixtures that comprise one or more sets of methylation-agnostic ratiometrically-encoded nucleic acid probes to produce detected target nucleic acid loci. A given set of methylation-agnostic ratiometrically-encoded nucleic acid probes in a given reaction mixture comprises a sufficient number of sequence permutations to detect substantially all epialleles present at a given target nucleic acid locus and in which the given set of methylation-agnostic ratiometrically-encoded nucleic acid probes comprises a predetermined ratio of differential labeling that produces a detectable signal that is sufficient to distinguish the detected epialleles present at the given target nucleic acid locus from epialleles detected at other target nucleic acid loci. The method also includes determining a methylation status of each epiallele detected at each detected target nucleic acid loci, thereby determining the methylation densities of the target nucleic acids in the sample. Optionally, other multiplexing approaches can also be used with the methods of the present disclosure.

In another aspect, this disclosure provides a method of determining a probability of a cancer in a subject. The method includes converting unmodified cytosine residues in nucleic acids from a sample obtained from the subject to uracil residues to produce converted nucleic acids, and amplifying the target nucleic acids among the converted nucleic acids in multiplex reaction mixtures that comprise one or more sets of methylation-agnostic ratiometrically-encoded nucleic acid probes to produce detected target nucleic acid loci. A given set of methylation-agnostic ratiometrically-encoded nucleic acid probes produces differential detectable signal signatures for each different target nucleic acid loci in the sample. The method also includes determining epiallelic methylation densities at each detected target nucleic acid locus and determining the probability of the cancer in the subject from the epiallelic methylation densities.

Various optional features of the above embodiments include the following. The target nucleic acids comprise at least about 10 different target nucleic acid loci, at least about 15 different target nucleic acid loci, at least about 25 different target nucleic acid loci, at least about 50 different target nucleic acid loci, at least about 75 different target nucleic acid loci, at least about 100 different target nucleic acid loci, or more different target nucleic acid loci. The target nucleic acid loci comprise cancer-specific methylation biomarkers. The method comprises forming droplets that comprise the reaction mixtures. The method comprises disposing the droplets in a microfluidic device. The microfluidic device comprises at least about 10,000 wells, at least about 50,000 wells, at least about 100,000 wells, at least about 200,000 wells, at least about 400,000 wells, at least about 600,000 wells, at least about 800,000 wells, at least about 1,000,000 wells, or more wells. The method comprises classifying the methylation status of each epiallele detected at each detected target nucleic acid loci by determining melting temperatures of the epialleles, determining melting curve shapes of the epialleles, and/or thresholding corresponding methylation density histograms. The amplifying step comprises a methylation-independent amplification technique. The method comprises diluting the reaction mixtures prior to amplifying the target nucleic acids among the converted nucleic acids such that the reaction mixtures each comprise at most about three epiallelic patterns. The method comprises quantifying substantially all unmethylated epialleles, substantially all heterogeneously-methylated epialleles, and substantially all fully-methylated epialleles in the sample. The sample comprises a cell-free DNA (cfDNA) sample. The sample comprises a Pap specimen, stool, sputum, urine, fresh tissue, formalin-fixed, paraffin-embedded (FFPE) tissue, blood, and/or cerebrospinal fluid (CSF).

Various additional optional features of the above embodiments include the following. The method comprises single-molecule sensitivity. The method comprises single-CpG-site resolution. The method produces absolute quantification over at least about five orders of magnitude. The method comprises a specificity of less than about 0.00005%. The method further comprises detecting and identifying at least one disease state from the detected target nucleic acid loci and/or from the methylation densities of the target nucleic acids. The disease state is detected and identified using a multivariate logistic regression model. The disease state comprises cancer and wherein the method further comprises identifying a tissue of origin of the cancer. The cancer comprises a cancer type selected from the group consisting of: bladder urothelial carcinoma (BLCA), breast invasive carcinoma (BRCA), cervical squamous cell carcinoma and endocervical adenocarcinoma (CESC), colon adenocarcinoma (COAD), esophageal carcinoma (ESCA), head and neck squamous cell carcinoma (HNSC), kidney renal clear cell carcinoma (KIRC), kidney renal papillary cell carcinoma (KIRP), acute myeloid leukemia (LAML), brain lower grade glioma (LGG), liver hepatocellular carcinoma (LIHC), lung adenocarcinoma (LUAD), lung squamous cell carcinoma (LUSC), pancreatic adenocarcinoma (PAAD), prostate adenocarcinoma (PRAD), skin cutaneous melanoma (SKCM), stomach adenocarcinoma (STAD), thyroid carcinoma (THCA), and uterine corpus endometrial carcinoma (UCEC). The method further comprises obtaining the sample from a subject. The method further comprises administering at least one therapy to the subject to treat the disease state. The reaction mixtures comprise one or more methylation-preferred nucleic acid primer pairs that amplify substantially all epiallelic fractions present at a given target nucleic acid locus irrespective of the methylation status of target nucleic acids comprising the given target nucleic acid locus in the sample. The differential labeling comprises at least a three-color (e.g., four-color, five-color, etc.) ratiometric fluorescence encoding technique. The methylation status of each epiallele detected at each detected target nucleic acid loci is determined using at least a four-color fluorescence-decoding digital high-resolution melt (dHRM) platform. The dHRM platform comprises a four-color imager matched with an HRM dye and three-color fluorescence encoding probes that decodes at least about 27 different ratiometric signatures. A given set of methylation-agnostic ratiometrically-encoded nucleic acid probes is labeled with fluorescent labels that produces distinct three-color fluorescence ratios when amplifying difference target nucleic acid loci. The sets of methylation-agnostic ratiometrically-encoded nucleic acid probes comprise pan-cancer-detecting probes and cancer-identifying probes. The methylation densities of the target nucleic acids comprise epigenetic signatures. The method comprises higher sensitivity to heterogeneous methylation than a digital methylation-specific PCR (dMSP) technique.

In another aspect, this disclosure provides a device, comprising a body structure that defines at least one inlet port, at least one fluidic channel, and at least one array of wells or droplets, which inlet port, fluidic channel, and array of wells or droplets fluidly communicate with one another, wherein the wells or droplets each comprise a multiplex reaction mixture that comprises: at most about one target nucleic acid, wherein unmodified cytosine residues in the target nucleic acid have been converted to uracil residues; one or more methylation-preferred nucleic acid primer pairs and/or one or more methylation-agnostic nucleic acid primer pairs that are configured to amplify substantially all epiallelic fractions present at a given target nucleic acid locus irrespective of the methylation status of target nucleic acids comprising the given target nucleic acid locus; one or more sets of methylation-agnostic ratiometrically-encoded nucleic acid probes, wherein a given set of methylation-agnostic ratiometrically-encoded nucleic acid probes is configured to produce differential detectable signal signatures for each different target nucleic acid loci in a sample when the target nucleic acid amplified and wherein the target nucleic acids in the sample comprise at least one of at least ten different target nucleic acid loci; wherein the target nucleic acids in the multiplex reaction mixtures are configured to be amplified in the array of wells or droplets to produce detected target nucleic acid loci; and wherein epiallelic methylation densities at each detected target nucleic acid loci are configured to be determined in the array of wells or droplets.

Various optional features of the above embodiments include the following. The target nucleic acids comprise at least about 10 different target nucleic acid loci, at least about 15 different target nucleic acid loci, at least about 25 different target nucleic acid loci, at least about 50 different target nucleic acid loci, at least about 75 different target nucleic acid loci, at least about 100 different target nucleic acid loci, or more different target nucleic acid loci. The target nucleic acid loci comprise cancer-specific methylation biomarkers. The device comprises at least about 10,000 wells or droplets, at least about 25,000 wells or droplets, at least about 50,000 wells or droplets, at least about 100,000 wells or droplets, at least about 200,000 wells or droplets, at least about 400,000 wells or droplets, at least about 600,000 wells or droplets, at least about 800,000 wells or droplets, at least about 1,000,000 wells or droplets, or more wells or droplets. A given set of methylation-agnostic ratiometrically-encoded nucleic acid probes is labeled with fluorescent labels that produces distinct three-color fluorescence ratios when amplifying difference target nucleic acid loci. The sets of methylation-agnostic ratiometrically-encoded nucleic acid probes comprise pan-cancer-detecting probes and cancer-identifying probes.

In another aspect, this disclosure provides a system, comprising a device having a body structure that defines at least one inlet port, at least one fluidic channel, and at least one array of wells or droplets, which inlet port, fluidic channel, and array of wells or droplets fluidly communicate with one another, wherein the wells or droplets each comprise a multiplex reaction mixture that comprises: at most about one target nucleic acid, wherein unmodified cytosine residues in the target nucleic acid have been converted to uracil residues; one or more methylation-preferred nucleic acid primer pairs and/or one or more methylation-agnostic nucleic acid primer pairs that are configured to amplify substantially all epiallelic fractions present at a given target nucleic acid locus irrespective of the methylation status of target nucleic acids comprising the given target nucleic acid locus; one or more sets of methylation-agnostic ratiometrically-encoded nucleic acid probes, wherein a given set of methylation-agnostic ratiometrically-encoded nucleic acid probes is configured to produce differential detectable signal signatures for each different target nucleic acid loci in a sample when the target nucleic acid amplified and wherein the target nucleic acids in the sample comprise at least one of at least ten different target nucleic acid loci; wherein the target nucleic acids in the multiplex reaction mixtures are configured to be amplified in the array of wells or droplets to produce detected target nucleic acid loci; and wherein epiallelic methylation densities at each detected target nucleic acid loci are configured to be determined in the array of wells or droplets. The system also includes at least one thermal modulator that is configured to thermocycle the multiplex reaction mixtures to amplify the target nucleic acids and to perform melting temperature analyses of amplified target nucleic acids in the array of wells or droplets; at least one detector that is capable of detecting detectable signals generated by the amplified target nucleic acids to produce the detected target nucleic acids and of detecting detectable signals corresponding to melting temperatures of the detected target nucleic acids to determine the epiallelic methylation densities at each detected target nucleic acid; and a controller operably connected to the thermal modulator and the detector, which controller is configured to modulate temperatures of the thermal modulator and to effect detection of the detectable signals from the detected target nucleic acids via the detector.

Various optional features of the above embodiments include the following. The target nucleic acids comprise at least about 10 different target nucleic acid loci, at least about 15 different target nucleic acid loci, at least about 25 different target nucleic acid loci, at least about 50 different target nucleic acid loci, at least about 75 different target nucleic acid loci, at least about 100 different target nucleic acid loci, or more different target nucleic acid loci. The target nucleic acid loci comprise cancer-specific methylation biomarkers. The device comprises at least about 10,000 wells or droplets, at least about 25,000 wells or droplets, at least about 50,000 wells or droplets, at least about 100,000 wells or droplets, at least about 200,000 wells or droplets, at least about 400,000 wells or droplets, at least about 600,000 wells or droplets, at least about 800,000 wells or droplets, at least about 1,000,000 wells or droplets, or more wells or droplets. A given set of methylation-agnostic ratiometrically-encoded nucleic acid probes is labeled with fluorescent labels that produces distinct three-color fluorescence ratios when amplifying difference target nucleic acid loci. The sets of methylation-agnostic ratiometrically-encoded nucleic acid probes comprise pan-cancer-detecting probes and cancer-identifying probes.

### BRIEF DESCRIPTION OF THE DRAWINGS

The accompanying drawings, which are incorporated in and constitute a part of this specification, illustrate certain embodiments, and together with the written description, serve to explain certain principles of the microfluidic devices, systems, kits, computer readable media, methods, and related aspects disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1. Detecting DNA methylation in early-stage cancers. (a) Unlike traditional PCR approaches (e.g., MSP), DREAMing employs digitization and unique primer design that provides sensitive and precise quantification of heterogeneously-methylated biomarkers (epialleles). (b) This key advantage enables detection of the aberrant stochastic DNA methylation patterns that arise at the early stages of carcinogenesis.

FIG. 2. Pan-cancer-detecting and Cancer-identifying Biomarkers. (a) Heatmap of TCGA-derived data, showing methylation β-values of 32 “pan-cancer-detecting” probes that exhibit widespread methylation in the vast majority of human cancers (colored bars). (b) Heatmap of phenotype-specific, “cancer-identifying” probes showing unsupervised clustering of 19 different human malignancies.

FIG. 3 DREAMing. (a) The DREAMing assay is based on the principle of methylation-sensitive high-resolution melt (MS-HRM), whereby the methylation density (% methylation) of epialleles can be determined by analyzing the melt temperature (Tm) of amplified bisulfite-converted templates. Unlike DREAMing, traditional MS-HRM in non-quantitative and is methylation statuses are obfuscated when performed in “real world” samples such as liquid biopsies that contain mixed epialleles. (b) DREAMing differs in that bisulfite converted DNA is first diluted down to a digital or “quasi-digital” level for PCR, followed by high-resolution melt to evaluate the methylation density status of methylation targets on a copy-by-copy basis, thereby allowing precise enumeration and quantification of all unmethylated (U), heterogeneously-methylated (HM) and fully-methylated (M) epialleles in the sample.

FIG. 4. Heterogeneous Methylation and the Detection of Cancer. In a recent blinded study investigating the use of stool-derived cfDNA for detection of the colorectal cancer methylation biomarker NDRG4, DREAMing demonstrated ~2.5× better clinical sensitivity than qMSP, while maintaining 100% specificity. Similar results have been achieved for other methylation biomarkers for the detection of lung and ovarian cancers using liquid biopsies.

FIG. 5. (a) Microfluidic DREAMing analysis of p14ARF synthetic sequences ranging in copy numbers of 0 to 1000 of each epiallelic species in 2 million unmethylated background molecules (i). (ii) A subset of melt curve derivatives from each serial dilution, color-coded by melt temperature, demonstrate methylation-independent amplification of all species as well as single-copy sensitivity. (iii) The digital heatmap reflects the methylation density of the template molecule in each well. (iv) The populations of the four epiallelic species can be quantified and assessed upon inspection and thresholding of the DREAM histogram. (b) Detected vs. Expected DNA copies. The expected copies per chip were calculated according to a Poisson distribution. The detected copy number represents the average for all methylation densities.

FIG. 6. EpiClass performance in liquid biopsies. ROC analysis of ZNF154 DREAMing data from validation cohort of 24 HGSC-positive and 12 control liquid biopsies. Curves compare the performance using thresholds identified by EpiClass using training data versus those based on mean methylation or heavy methylation only (MSP).

FIG. 7. REM-DREAMing: a cost-effective solution for pan-cancer screening and identification. The REM-DREAMing comprises novel innovations. (i) cfDNA from liquid biopsies is extracted and undergoes bisulfite conversion. (ii) The sample is split and combined with REM-DREAMing reagents for two, 27-plex assays, which include R:O:Y ratiometrically-encoded methylation-agnostic probes and EvaGreen DNA-binding dye. (iii) The two resulting solutions are respectively digitized in 400,000-picowell modules on the microfluidic chip, which is then placed on a thermal cycler for PCR amplification. (iv) Amplified targets are first identified by their unique 3-color fluorometric signature (here 2:2:2=Target 27), followed by dHRM to assess epiallelic methylation density (here medium-density) of each respective target-template. (v) Methylation data from all positive picowells are tallied to create a DREAM analysis histogram which is evaluated by multivariate logistic regression to determine patient cancer status and, if positive, predicted tissue of origin.

FIG. 8. Overview of algorithm for in silico design and selection of primer pools for multiplex DREAMing assays. Our primer design algorithm uses our custom design criteria to first identify DREAMing-suitable (DS) primer candidates for all target loci in the multiplex assay. DS-primers for each target are then screened against each other using established thermodynamic principles to identify pools of minimally-interacting (MI) primer pairs. Pools for each target are then screened against other to identify paired MI-multiplex (MIM) pools, each targeting 25 targets and 2 control loci.

FIG. 9. In silico primer design for highly multiplexed methylation assays. Post-PCR Bioanalyzer traces of independent (a) 100-plex and (b) 99-plex assays showing amplification of 240-290 bp BSC genomic DNA targets (green boxes) with no appreciable primer-dimer formation.

FIG. 10. Methylation-agnostic Probes With Fluorometric signatures. (a) Agnostic probes are designed with “wobble-bases” that detect all methylation patterns of bisulfite converted (BSC) DNA amplicons. In contrast, traditional MSP probes are designed to detect only a specific methylation pattern (i.e. fully methylated DNA). (b) Each target is detected by a set of 3 agnostic TaqMan probes of identical sequences that are differentially labeled with red, orange or yellow fluorophore. Probes for each respective target are added at predefined color ratio designated for each specific target. (c) Example of detecting Target 3 using a set of Agnostic probe 3 pre-mixed at a ratio of 1:1:2 (R:O:Y). Post digital PCR, the digital microchamber measured with a fluorescence ratio (R:O:Y) close to 1:1:2 indicates the presence of the Target 2. The methylation status of target is determined by the melt temperature, Tm. (d) Demonstration of a 6-plex assay for UTI bacteria based on 2-color ratiometric encoding70. (e) Intensity scatterplot and micrograph (inset) showing 2-color ratiometric decoding in a digital microfluidic device.

FIG. 11. REM-DREAMing assay principle. (a) A multi-unit microfluidic chip is used to digitize DNA samples containing rare epialleles and for parallel processing of multiple samples. (b) The chip is then placed on an instrument integrated with thermal control and 4-color, real-time imager for digital PCR and high resolution melt (HRM). (c) During PCR, fluorescence is generated from EvaGreen and red, orange and yellow agnostic probes. For each positive digital microchamber, the target is first identified by its characteristic red-orange yellow (R:O:Y) fluorescence intensity. Subsequent HRM analysis via EvaGreen fluorescence is used to determine the methylation status of the respective target.

FIG. 12. Microfluidic chip fabrication. (a) A microfluidic array chip for partitioning DNA into picoliter-sized side chambers for digital PCR and HRM. (b) A CAD schematic and image of the actual device that is fabricated using a simple single layer soft lithography approach.

FIG. 13. An instrument integrated with thermal cycler and high-resolution imager for real-time fluorescence measurements.

FIG. 14. Schematic of method of determining methylation densities of target nucleic acids in a Pap sample.

FIGS. 15A-15E. Schematic steps of method of determining methylation densities of target nucleic acids in a sample using a microfluidic device.

FIG. 16. Exemplary plots of methylation statuses of various nucleic acid targets.

FIG. 17. Exemplary plots of methylation densities of various nucleic acid targets.

FIGS. 18A-18F. Exemplary plots showing the detection of various epialleles.

FIGS. 19A and 19B. Exemplary plots showing methylation thresholds for various biomarker combinations.

FIGS. 20A-20H. Exemplary plots showing analysis for both epiallelic copy number and epiallelic fraction in samples.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, cranial implant devices, and component parts, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administering: As used herein, the terms “administering” and “administration” refer to any method of providing a pharmaceutical preparation or other treatment to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

Amplifying: As used herein, “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using RT-PCR is a form of amplification. Furthermore, the generation of multiple RNA molecules from a single DNA molecule during the process of transcription is also a form of amplification.

Cancer Type: As used herein, “cancer,” “cancer type” or “tumor type” refers to a type or subtype of cancer defined, e.g., by histopathology. Cancer type can be defined by any conventional criterion, such as on the basis of occurrence in a given tissue (e.g., blood cancers, central nervous system (CNS), brain cancers, lung cancers (small cell and non-small cell), skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers), unknown primary origin and the like, and/or of the same cell lineage (e.g., carcinoma, sarcoma, lymphoma, cholangiocarcinoma, leukemia, mesothelioma, melanoma, or glioblastoma) and/or cancers exhibiting cancer markers, such as Her2, CA15-3, CA19-9, CA-125, CEA, AFP, PSA, HCG, hormone receptor and NMP-22. Cancers can also be classified by stage (e.g., stage 1, 2, 3, or 4) and whether of primary or secondary origin.

Cell-Free Nucleic Acid: As used herein, “cell-free nucleic acid” refers to nucleic acids not contained within or otherwise bound to a cell or, in some embodiments, nucleic acids remaining in a sample following the removal of intact cells. Cell-free nucleic acids can include, for example, all non-encapsulated nucleic acids sourced from a bodily fluid (e.g., blood, plasma, serum, urine, cerebrospinal fluid (CSF), etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell death processes, e.g., cellular necrosis, apoptosis, or the like. Cell-free nucleic acids can be found in an efferosome or an exosome. Some cell-free nucleic acids are released into bodily fluid from cancer cells, e.g., circulating tumor DNA (ctDNA). Others are released from healthy cells. CtDNA can be non-encapsulated tumor-derived fragmented DNA. Another example of cell-free nucleic acids is fetal DNA circulating freely in the maternal blood stream, also called cell-free fetal DNA (cffDNA). A cell-free nucleic acid can have one or more epigenetic modifications, for example, a cell-free nucleic acid can be acetylated, 5-methylated, ubiquitylated, phosphorylated, sumoylated, ribosylated, and/or citrullinated.

Cellular Origin: As used herein, “cellular origin” in the context of cell-free nucleic acids means the cell type from which a given cell-free nucleic acid molecule derives or otherwise originates (e.g., via a apoptotic process, a necrotic process, or the like). In certain embodiments, for example, a given cell-free nucleic acid molecule may originate from a tumor cell (e.g., a cancerous pulmonary cell, etc.) or a non-tumor or normal cell (e.g., a non-cancerous pulmonary cell, etc.).

Detect: As used herein, “detect,” “detecting,” or “detection” refers to an act of determining the existence or presence of one or more target nucleic acids (e.g., nucleic acids having targeted mutations or other markers) in a sample.

Detectable Signal: As used herein, “detectable signal” refers to signal output at an intensity or power sufficient to be detected in a given detection system. In certain embodiments, a detectable signal is emitted from a label (e.g., a fluorescent label or the like) associated with a given primer nucleic acid and/or probe nucleic acid.

Epigenetic Information: As used herein, “epigenetic information” in the context of a DNA polymer means one or more epigenetic patterns exhibited in that polymer.

Epigenetic Locus: As used herein, “epigenetic locus” or “epigenetic site” means a fixed position on a chromosome that exhibits different states or statuses that do not involve changes or alterations in nucleotide sequence. For example, a given epigenetic locus may or may not be acetylated, methylated (e.g., modified with 5-methylcytosine (5mC), modified with 5-hydroxymethylcytosine (5hmC), and/or the like), ubiquitylated, phosphorylated, sumoylated, ribosylated, citrullinated, have a histone post-translational modification or other histone variation, and/or the like.

Epigenetic Pattern: As used herein, “epigenetic pattern” means an epigenetic state or status exhibited by one or more epigenetic loci in a given DNA molecule. For example, DNA molecules or cfDNA fragments that comprise a given genomic region or locus (e.g., a CTCF binding region, etc.) may also exhibit epigenetic patterns in which some of those DNA molecules include a certain number of epigenetic loci that are methylated, whereas in other instances corresponding epigenetic loci in other DNA molecules or cfDNA fragments that comprise the same genomic region are unmethylated.

Exonuclease Probe: As used herein, “exonuclease probe” refers to a labeled oligonucleotide that is capable of producing a detectable signal change upon being cleaved. To illustrate, in certain embodiments an exonuclease probe is a 5′-nuclease probe comprising two labeling moieties and emits radiation of increased intensity after one of the labels is cleaved or otherwise separated from the oligonucleotide. In some of these embodiments, for example, the 5′-nuclease probe is labeled with a 5′ terminus quencher moiety and a reporter moiety at the 3′ terminus of the probe. In certain embodiments, 5′-nuclease probes are labeled at one or more positions other than, or in addition to, these terminal positions. When the probe is intact, energy transfer typically occurs between the labeling moieties such that the quencher moiety at least in part quenches the fluorescent emission from the acceptor moiety. During an extension step of a polymerase chain reaction, for example, a 5′-nuclease probe bound to a template nucleic acid is cleaved by the 5′ to 3′ nuclease activity of, e.g., a Taq polymerase or another polymerase having this activity such that the fluorescent emission from the acceptor moiety is no longer quenched. To further illustrate, in certain embodiments 5′-nuclease probes include regions of self-complementarity such that the probes are capable of forming hairpin structures under selected conditions. In these embodiments, 5′-nuclease probes are also referred to herein as “hairpin probes.”

Hairpin Probe: As used herein, “hairpin probe” refers to an oligonucleotide that can be used to effect target nucleic acid detection and that includes at least one region of self-complementarity such that the probe is capable of forming a hairpin or loop structure under selected conditions. Typically, hairpin probes include one or more labeling moieties. In one exemplary embodiment, quencher moieties and reporter moieties are positioned relative to one another in the hairpin probes such that the quencher moieties at least partially quench light emissions from the reporter moieties when the probes are in hairpin confirmations. In contrast, when the probes in these embodiments are not in hairpin confirmations (e.g., when the probes are hybridized with target nucleic acids), light emissions the acceptor reporter moieties are generally detectable. Hairpin probes are also known as molecular beacons in some of these embodiments. Hairpin probes can also function as 5′-nuclease probes or hybridization probes in certain embodiments.

Hybridization Probe: As used herein, “hybridization probe” refers an oligonucleotide that includes at least one labeling moiety that can be used to effect target nucleic acid detection. In some embodiments, hybridization probes function in pairs. In some of these embodiments, for example, a first hybridization probe of a pair includes at least one donor moiety at or proximal to its 3′-end, while the second hybridization probe of the pair includes at least one acceptor moiety (e.g., LC-Red 610, LC-Red 640, LC-Red 670, LC-Red 705, JA-270, CY 5, or CY 5.5) at or proximal to its 5′-end. The probes are typically designed such that when both probes hybridize with a target or template nucleic acid (e.g., during a PCR), the first hybridization probe binds to the 5′-end side or upstream from the second hybridization probe and within sufficient proximity for energy transfer to occur between the donor and acceptor moieties to thereby produce a detectable signal. Typically, the second hybridization probe also includes a phosphate or other group on its 3′-end to prevent extension of the probe during a PCR.

Methylation-agnostic ratiometrically-encoded nucleic acid probe: As used herein, “methylation-agnostic ratiometrically-encoded nucleic acid probe” refers to nucleic acid probes that bind to CpG-rich loci of a specific target nucleic acid independent of the methylation pattern exhibited by that target nucleic acid.

Methylation-preferred nucleic acid primer pair. As used herein, “methylation-preferred nucleic acid primer pair” refers to a nucleic acid primer pair that enable the detection of all epiallelic fractions, regardless of their methylation patterns.

Label: As used herein, “label” refers to a moiety attached (covalently or non-covalently), or capable of being attached, to a molecule, which moiety provides or is capable of providing information about the molecule (e.g., descriptive, identifying, etc. information about the molecule). Exemplary labels include donor moieties, acceptor moieties, fluorescent labels, non-fluorescent labels, calorimetric labels, chemiluminescent labels, bioluminescent labels, radioactive labels, mass-modifying groups, antibodies, antigens, biotin, haptens, and enzymes (including, e.g., peroxidase, phosphatase, etc.).

Mixture: As used herein, “mixture” refers to a combination of two or more different components.

Nucleic Acid: As used herein, “nucleic acid” refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., bromodeoxyuridine (BrdU)), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA, cfDNA, ctDNA, or any combination thereof.

Nucleic Acid Primer. As used herein, “nucleic acid primer” or “primer” refers to a nucleic acid that can hybridize to a target or template nucleic acid and permit chain extension or elongation using, e.g., a nucleotide incorporating biocatalyst, such as a polymerase under appropriate reaction conditions. A primer nucleic acid is typically a natural or synthetic oligonucleotide (e.g., a single-stranded oligodeoxyribonucleotide). Although other primer nucleic acid lengths are optionally utilized, they typically comprise hybridizing regions that range from about 8 to about 100 nucleotides in length. Short primer nucleic acids generally require cooler temperatures to form sufficiently stable hybrid complexes with template nucleic acids. A primer nucleic acid that is at least partially complementary to a subsequence of a template nucleic acid is typically sufficient to hybridize with the template for extension to occur. A primer nucleic acid can be labeled, if desired, by incorporating a label detectable by, e.g., spectroscopic, photochemical, biochemical, immunochemical, chemical, or other techniques. To illustrate, useful labels include donor moieties, acceptor moieties, quencher moieties, radioisotopes, electron-dense reagents, enzymes (as commonly used in performing ELISAs), biotin, or haptens and proteins for which antisera or monoclonal antibodies are available. Many of these and other labels are described further herein and/or are otherwise known in the art. One of skill in the art will recognize that, in certain embodiments, primer nucleic acids can also be used as probe nucleic acids.

Nucleic Acid Probe: As used herein, “nucleic acid probe” or “probe” refers to a labeled or unlabeled oligonucleotide capable of selectively hybridizing to a target or template nucleic acid under suitable conditions. Typically, a probe is sufficiently complementary to a specific target sequence contained in a nucleic acid sample to form a stable hybridization duplex with the target sequence under a selected hybridization condition, such as, but not limited to, a stringent hybridization condition. A hybridization assay carried out using a probe under sufficiently stringent hybridization conditions permits the selective detection of a specific target sequence. The term “hybridizing region” refers to that region of a nucleic acid that is exactly or substantially complementary to, and therefore capable of hybridizing to, the target sequence. For use in a hybridization assay for the discrimination of single nucleotide differences in sequence, the hybridizing region is typically from about 8 to about 100 nucleotides in length. Although the hybridizing region generally refers to the entire oligonucleotide, the probe may include additional nucleotide sequences that function, for example, as linker binding sites to provide a site for attaching the probe sequence to a solid support. A probe of the invention is generally included in a nucleic acid that comprises one or more labels (e.g., donor moieties, acceptor moieties, and/or quencher moieties), such as exonuclease probe (e.g., a 5′-nuclease probe), a hybridization probe, a fluorescent resonance energy transfer (FRET) probe, a hairpin probe, or a molecular beacon, which can also be utilized to detect hybridization between the probe and target nucleic acids in a sample. In some embodiments, the hybridizing region of the probe is completely complementary to the target sequence. However, in general, complete complementarity is not necessary (i.e., nucleic acids can be partially complementary to one another); stable hybridization complexes may contain mismatched bases or unmatched bases. Modification of the stringent conditions may be necessary to permit a stable hybridization complex with one or more base pair mismatches or unmatched bases. Stability of the target/probe hybridization complex depends on a number of variables including length of the oligonucleotide, base composition and sequence of the oligonucleotide, temperature, and ionic conditions. One of skill in the art will recognize that, in general, the exact complement of a given probe is similarly useful as a probe. One of skill in the art will also recognize that, in certain embodiments, probe nucleic acids can also be used as primer nucleic acids.

Reaction Mixture: As used herein, “reaction mixture” refers a mixture that comprises molecules that can participate in and/or facilitate a given reaction or assay. To illustrate, an amplification reaction mixture generally includes a solution containing reagents necessary to carry out an amplification reaction, and typically contains primers, a biocatalyst (e.g., a nucleic acid polymerase, a ligase, etc.), dNTPs, and a divalent metal cation in a suitable buffer. A reaction mixture is referred to as complete if it contains all reagents necessary to carry out the reaction, and incomplete if it contains only a subset of the necessary reagents. It will be understood by one of skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of skill in the art that reaction components are packaged separately for commercialization and that useful commercial kits may contain any subset of the reaction or assay components.

Sample: As used herein, “sample” refers to a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a nucleic acid), which is assayed as described herein. A sample may also be any body fluid or excretion (for example, but not limited to, blood, urine, stool, saliva, tears, bile) that contains cells, cell components, or non-cellular fractions.

Sensitivity: As used herein, “sensitivity” in the context of a given assay or method refers to the ability of the assay or method to detect and distinguish between targeted (e.g., cfDNA fragments originating from tumor cells) and non-targeted (e.g., cfDNA fragments originating from non-tumor cells) analytes.

Specificity: As used herein, “specificity” in the context of a diagnostic analysis or assay refers to the extent to which the analysis or assay detects an intended target analyte to the exclusion of other components of a given sample.

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.” For example, a subject can be an individual who has been diagnosed with having a cancer, is going to receive a cancer therapy, and/or has received at least one cancer therapy. The subject can be in remission of a cancer.

System: As used herein, “system” in the context of analytical instrumentation refers a group of objects and/or devices that form a network for performing a desired objective.

Target: As used herein, “target” refers to a biomolecule (e.g., a nucleic acid, etc.), or portion thereof, that is to be amplified, detected, and/or otherwise analyzed.

Threshold: As used herein, “threshold” refers to a separately determined value used to characterize or classify experimentally determined values.

Treatment: As used herein, “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human.

Value: As used herein, “value” generally refers to an entry in a dataset that can be anything that characterizes the feature to which the value refers. This includes, without limitation, numbers, words or phrases, symbols (e.g., + or −) or degrees.

### DETAILED DESCRIPTION

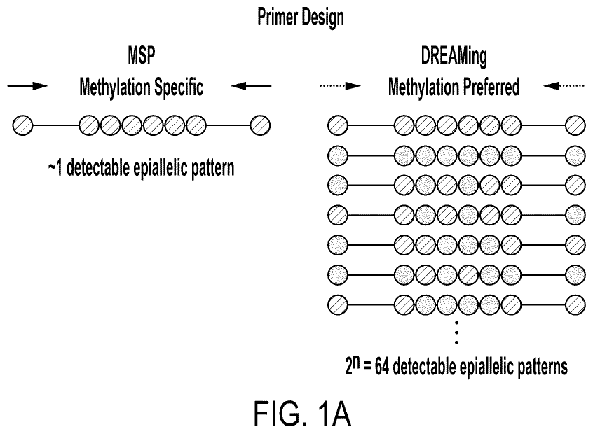

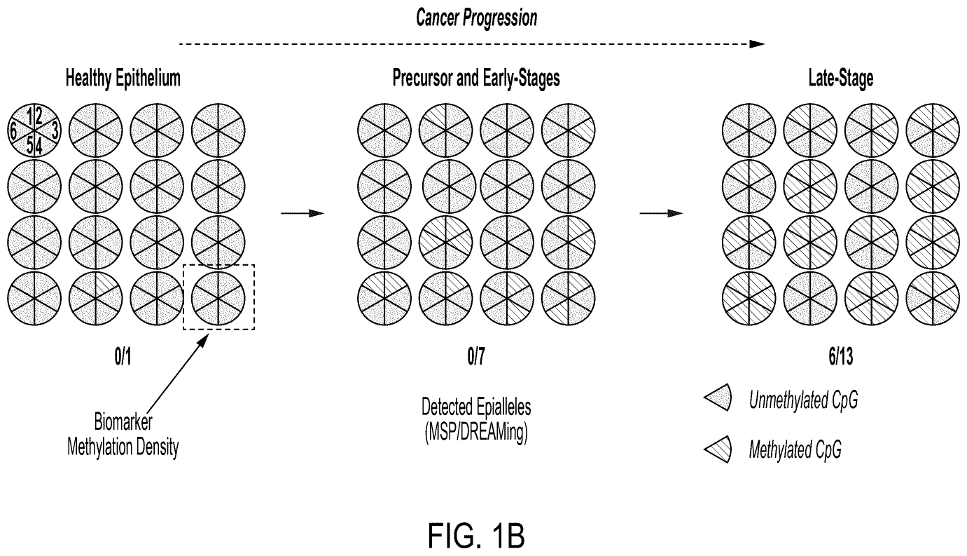

This application discloses techniques, sometimes referred to herein as DREAMing (Discrimination of Rare EpiAlleles by Melt), that utilize unique primer designs and precise, high-resolution melt curve analysis to distinguish and enumerate individual copies of epiallelic species at single-CpG site resolution in fractions as low as 0.005% directly from liquid biopsies, among other sample types. The methods can be implemented using microwell plates, but can be readily incorporated into massively parallel microfluidic devices to achieve unprecedented levels of analytical specificity (0.00005%) and single-copy sensitivity at or near single-CpG-site resolution. In some embodiments, for example, the methods disclosed herein couple ultra-high density digitization with a novel, high-dimensional multiplexing strategy to create a digital microfluidic platform for rapid, simultaneous methylation assessment and quantification of 50+ plex methylation biomarker panels for inexpensive, early detection and identification of early-stage cancers. Example microfluidic devices and related methods that can be adapted for use with the present disclosure are provided in International Publication No. WO 2021/003301, which is incorporated by reference. To illustrate, FIG. 1 provides a comparison of aspects traditional methylation-specific PCR approaches to the DREAMing methods of the present disclosure.

The detection of early-stage cancers, for examples, by methylation analysis of cfDNA needs a platform that is not only sensitive, but also specific, being able to discriminate extremely-rare (<<0.1%) heterogeneous methylation patterns within a large background of healthy-tissue DNA. The microfluidic DREAMing platforms disclosed herein (sometimes referred to as “μ-DREAMing”) combines massively parallel sample digitation, digital PCR and precise melt curve analysis to distinguish and enumerate individual copies of epiallelic species at or near single-CpG-site resolution. These attributes enable the ability to discriminate a single methylated DNA fragment among ~2,000,000 wild-type DNA fragments and can provide orders of magnitude higher sensitivity to heterogeneous methylation than “gold-standard” dMSP assays. μ-DREAMing uses a simple workflow and instrumentation similar to a PCR test, thereby providing a facile, highly sensitive and inexpensive means of detecting rare methylation patterns associated with early-stage cancers, among other disease states.

The inability to achieve high degrees of multiplexing has been a limitation of otherwise attractive dPCR analysis techniques. The methods of the present disclosure overcome this limitation using a ratiometric fluorescence detection scheme. The methods of the present disclosure are compatible with standard TaqMan or hybridization probe chemistry and allow both simultaneous identification and melt analysis of many epiallelic gene targets. The probes themselves are designed using a novel “methylation-agnostic” design to allow for binding to the CpG-rich loci of a specific target independent of the methylation patterns. Multiple pairs of agnostic probes of distinct three-color fluorescence ratios targeting at the selected gene panel are used in the assay, allowing each DNA target to be identified by its corresponding fluorescence ratio upon dPCR with its methylation status revealed by dHRM.

While there has undoubtedly been significant progress in the development of targeted-sequencing-based methods for the analysis of cfDNA, many of these approaches are expensive (>$1000 per sample), elaborate and require the use of proprietary algorithms that currently preclude widespread adoption both in the clinic and research settings. In contrast, the methods disclosed herein are based on dHRM on a disposable array chip, with a simple workflow similar to qPCR and an anticipated cost of less than $50 per 50-plex assay. The digital, absolutely quantitative nature of the methods of the present disclosure facilitates standardization by enabling the use of clear, quantitative thresholds for assessing patient samples. Furthermore, in some embodiments, the combination of both “pan-cancer-detecting” and phenotype-specific, “cancer-identifying” methylation biomarkers affords the potential to not only detect cancer, but also predict the tissue of origin.

### Universal Cancer Detection Using DNA Methylation Biomarkers

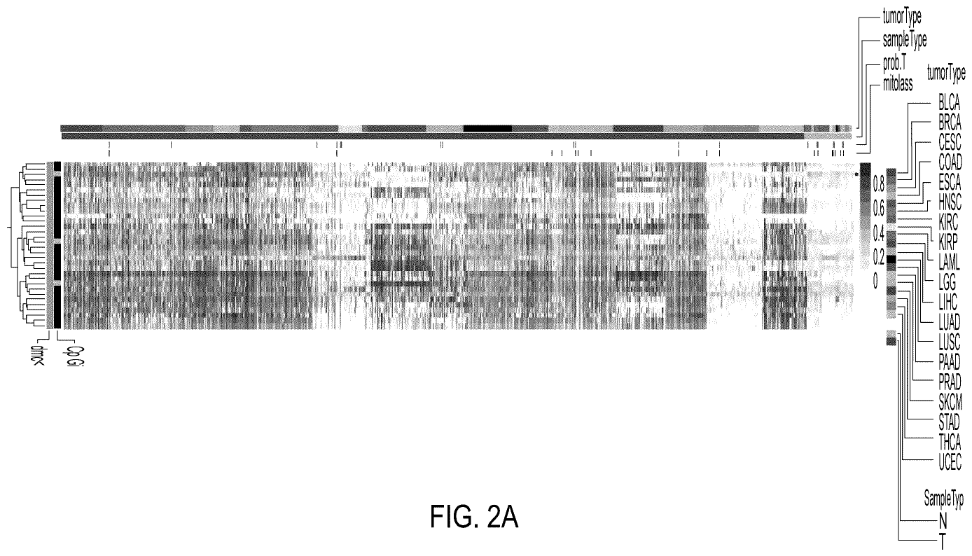

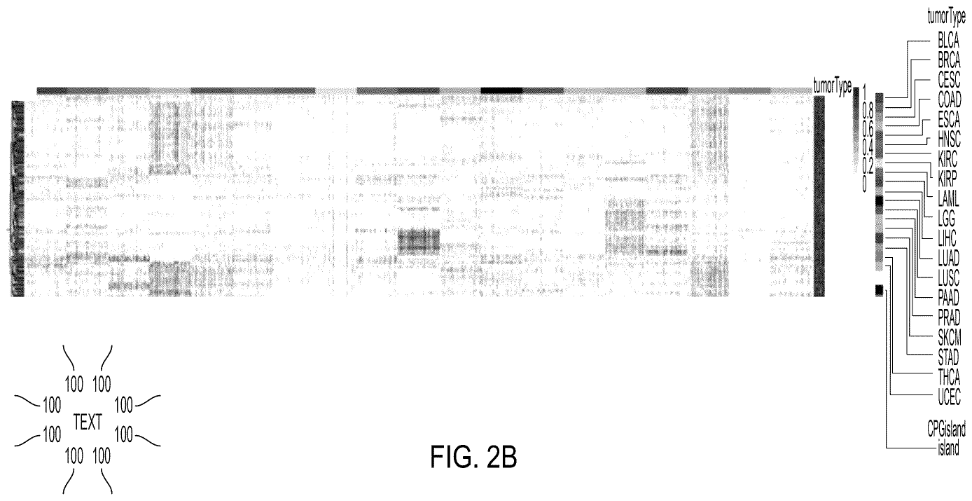

Numerous studies have now provided evidence that the vast majority of human cancers can be detected through analysis of cfDNA derived from a simple blood draw. In order to fully leverage this knowledge for pan-cancer diagnostics, a prospective test would ostensibly need to identify a panel of biomarkers that are universally present in most forms of cancer and, ideally, also enable identification of tumor tissues of origin. To determine whether methylation loci might be used for such an application, the present disclosure adapted successful biomarker selection algorithms to identify a biomarker panel for sensitive and accurate detection of the five most-prevalent cancers (core cancers): lung squamous cell carcinoma (LUSC) and adenocarcinoma (LUAD), breast, colon, and prostate cancer. These five cancers represent the leading causes of cancer death in the United States. Analyzing Infinium 450K genome-wide methylation data from these cancers (2,281 tumor samples) and 255 matched normal samples, 343 hypermethylated CpG probes (regions) were identified that demonstrated discriminative capacity for cancer vs. non-cancer. From this panel, using a feature selection approach, 32 probe-regions were identified with little or no methylation in normal tissues (needed for cancer-specific methylation detection), but were methylated in nearly all tumors. These 32 “pan-cancer-detecting” probes correspond to 27 genomic regions (5 regions were identified with 2 different probes). We examined whether these 32 probes also have cancer-specific DNA methylation in other malignancies. In 19 tumor types examined in TCGA (>6,000 tumors), we observed that these DNA methylation changes, present in LUSC, LUAD, breast, colon, and prostate cancers, were also methylated in nearly all other cancer histologies, including ovarian, endometrial and pancreatic cancers (FIG. 2a). Overall, the pan-cancer loci exhibited a clinical sensitivity of 91% and a clinical specificity of 98%. We also sought to determine if changes in DNA methylation at other loci could be used to determine the tissue of origin. Using the Infinium 450K genome-wide methylation data from all common cancer types, as well as matched normal tissues from these organs, we identified a panel of hypermethylated phenotype-specific, “cancer identifying”probes that can discriminate 19 different TCGA tumor types (including cancer types targeted by the proposed REM-DREAMing assay: lung, breast, colon, pancreas and endometrial cancers), with clearly distinct methylation phenotypes in each cancer type (FIG. 2b). These data demonstrate the unique epigenetic signature of hypermethylated loci, which, when coupled with performance of “pan-cancer-detecting” loci, have the potential to provide a simple approach to not only detect the vast majority of cancers, but also the ability to reliably identify respective cancer tissues of origin.

### DREAMing—Simple, Inexpensive and Highly Sensitive Analysis of Heterogeneous Methylation

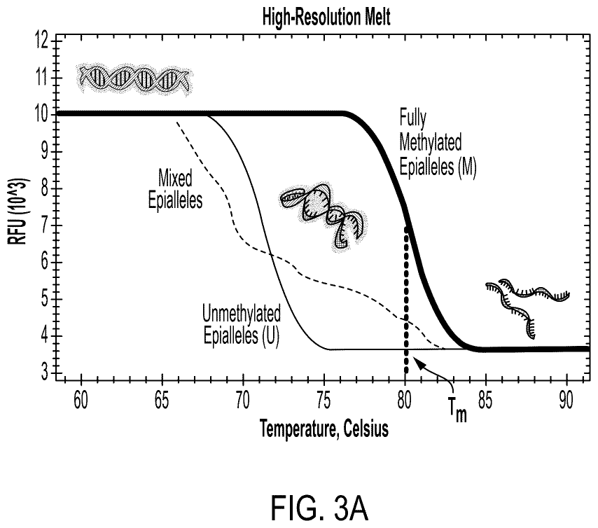

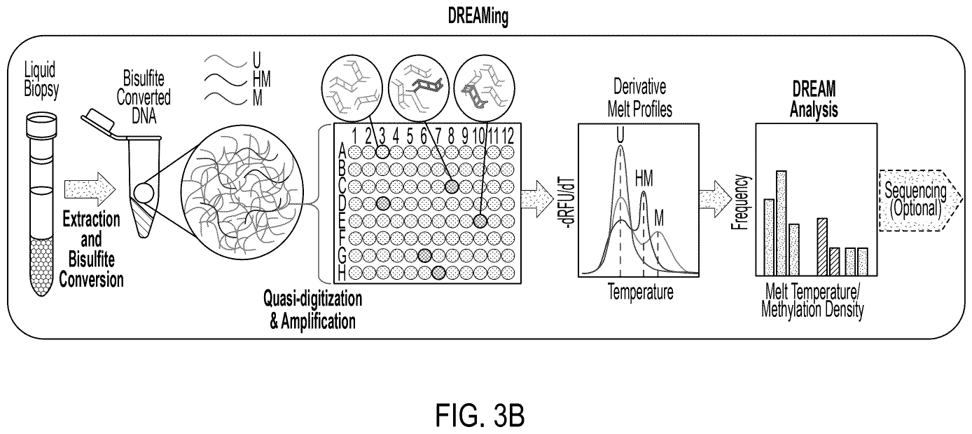

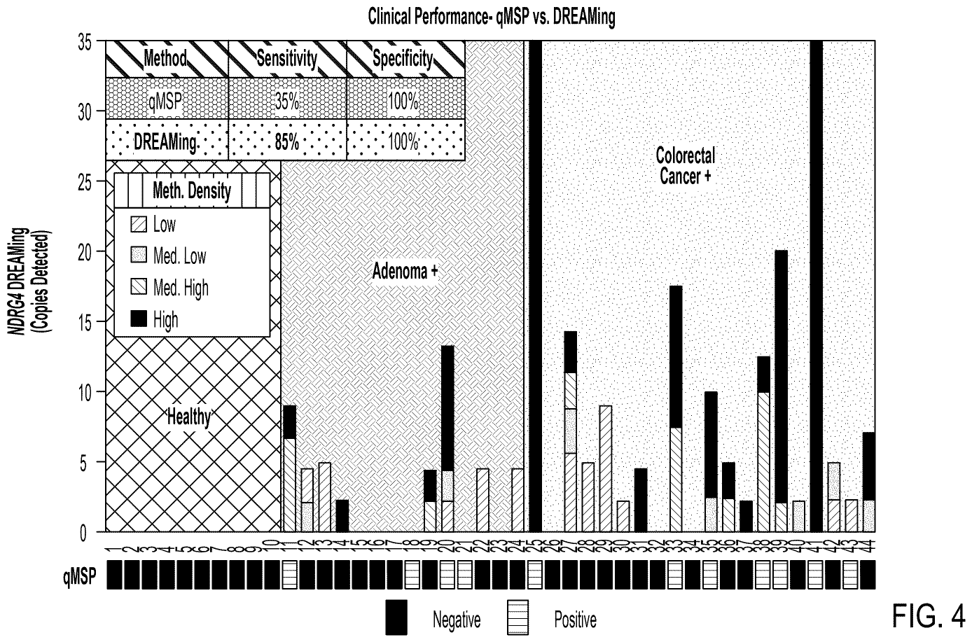

The digital epigenetic detection approaches disclosed herein provide a facile and inexpensive means ideally suited to the detection and assessment of heterogeneous methylation within ultra-rare epiallelic variants. DREAMing was originally demonstrated for use with standard real-time PCR instruments, and leveraged semi-limiting dilution scheme and precise high-resolution melt curve analysis to allow enumeration of bisulfite converted (BSC) heterogeneously-methylated DNA at single-CpG-site resolution and single-copy sensitivity (FIG. 3). One of the key design principles of DREAMing is the use of “methylation-preferred” primers, which, unlike so-called methylation-specific primers, enables detection of all epiallelic fractions, regardless of their methylation pattern, at analytical specificities of 1 in 20,000 or better, typically allowing detection of 3-to-30-fold more epialles in clinical samples than qMSP. Second, unlike qMSP, DREAMing provides absolute quantification of targets and assessment of methylation density on a copy-by-copy basis, which can then be plotted as a “DREAM analysis” of the epigenetic heterogeneity for each sample-biomarker-locus. These crucial advantages enable single copies of even partially-methylated DNA to be detected and enumerated, providing a substantial improvement over qMSP in analytical sensitivity for cancer diagnostics. This improved analytical sensitivity can directly translate into dramatic improvements in clinical sensitivity. For example, in a head-to-head comparison of detection of the colorectal cancer methylation biomarker, NDRG4, in DNA extracted from stool samples DREAMing demonstrated over 2.3-fold better clinical sensitivity than qMSP, while at the same time maintaining absolute (100%) specificity (FIG. 4).

### Microfluidic DREAMing (μ-DREAMing)

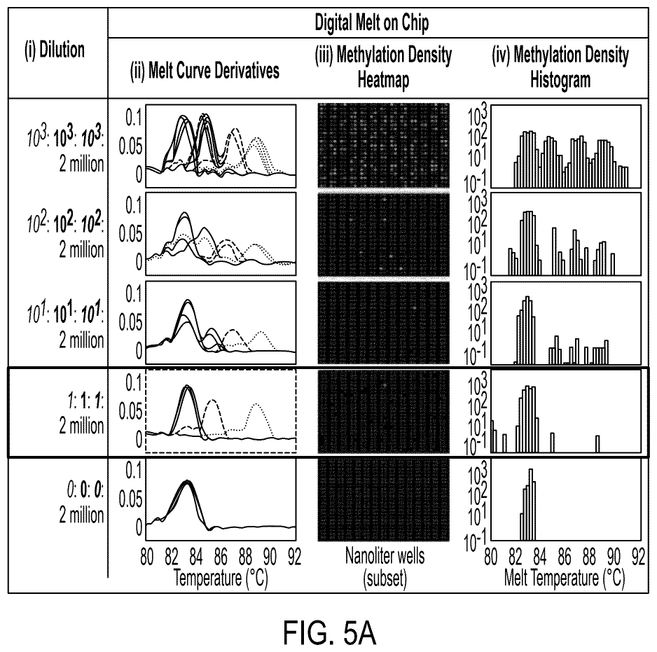

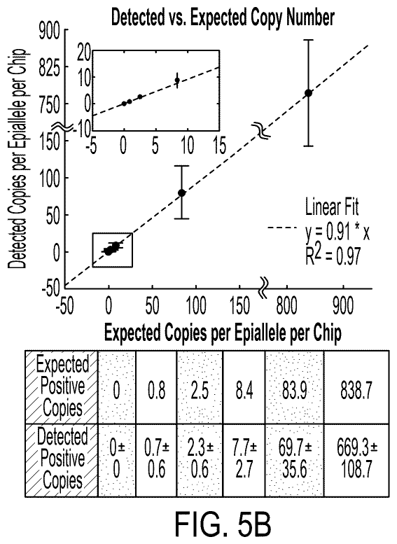

Some embodiments provide a microfluidic device that consists of a 4096 nanoliter-well static array and provides the ability to perform DREAMing high-resolution melt in a highly parallelized manner. To assess the capabilities of this “μ-DREAMing” platform, synthetic targets representative of various bisulfite-converted sequences of the tumor suppressor gene, CDKN2A (p14ARF), were used as a model system. Four methylation densities were analyzed: 0%, 33%, 67% and 100%. The unmethylated (0%) sequence represented the background population and was set to 500 copies per nL (2 million copies overall). The three methylated variants were digitized on chip, amplified and identified by DREAMing. The detected number of targets for each epiallele was calculated by the number of positive melt-discriminated wells while accounting for a Poisson distribution. A serial dilution of methylated variants at concentrations of 0, 1, 10, 100, and 1000 copies/chip was performed in the presence of the 2 million unmethylated epialleles (FIG. 5a, i). Representative traces of fluorescence signals from individual wells are shown for each test (FIG. 5a, ii), which were classified by melt temperature via thresholding and color-coded by methylation density. Once discriminated by melt temperature, the digital result was converted to a methylation density heatmap (FIG. 5a, iii), which was then further quantified and analyzed via a methylation heterogeneity histogram (FIG. 5a, iv). The platform demonstrated absolute quantitation, as the calculated number of copies closely matched the expected number (FIG. 5b). The results of this analysis demonstrate that the microfluidic DREAMing platform provides absolute quantitation over five orders of magnitude, while maintaining accurate HRM-based detection down to single methylated variant among 2 million wild-type epialleles, or 0.00005%. Even higher specificities are ostensibly achievable by the microfluidic DREAMing platform as it is only limited by the appropriate dynamic range of the application. Importantly, the entire microchip costs under one dollar to make in quantity and uses less than 2% of the reagents/costs compared to the 96-well DREAMing format.

### Epiallelic Methylation Classifier (EpiClass)

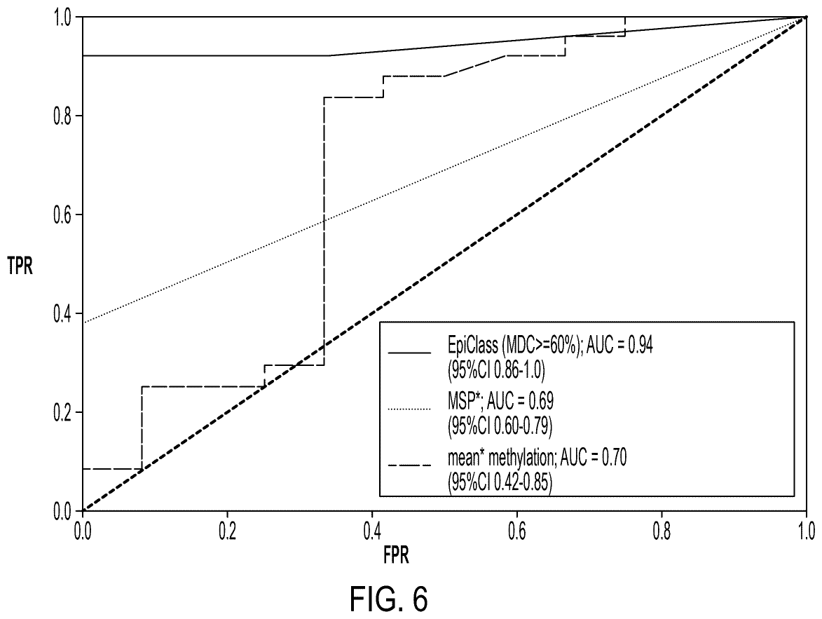

Some embodiments utilize a biostatistical tool called “EpiClass” that can be used to optimize the clinical performance of DREAMing by leveraging statistical differences in single-molecule sample methylation density distributions51. EpiClass utilizes tabularized DREAMing data to calculate the true positive and false positive rates as determined by iteratively varying methylation density and epiallelic fraction cutoffs. Overall, EpiClass can greatly improve the diagnostic performance of methylation biomarkers, particularly in difficult samples such as liquid biopsies that contain only small fractions of tumor DNA (FIG. 6). In the present disclosure, we implement and expand the EpiClass approach to identify cutoffs for a panel of markers in order to optimize the performance of multivariate logistic regression algorithms for the detection and identification of common human cancers.

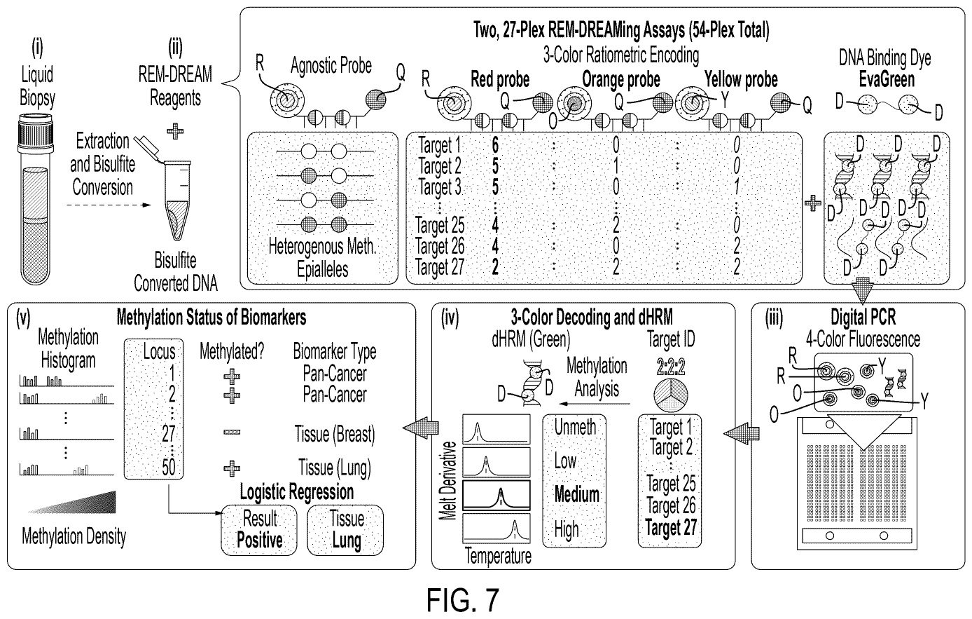

In some embodiments, the present disclosure provides a low-cost, digital microfluidic platform for early detection and identification of multiple cancer types by parallelized, dHRM-based DNA methylation analysis. REM-DREAMing features high-dimensional ratiometric encoding with novel, “methylation agnostic” probes to enable simultaneous detection and assessment of 50 methylation biomarkers per disposable chip (FIG. 7). The panel of 50 methylation biomarkers are carefully selected based on their ability to provide both detection and accurate identification of six prevalent cancer types (lung, colon, endometrial, ovarian, breast and pancreatic) from heterogeneous liquid biopsy samples. We design and analytically validate two REM-DREAMing primer pools, each targeting 27 loci (25 biomarkers+2 control). Each REM-DREAMing target is then paired with a unique, methylation-agnostic probe and corresponding 3-color ratiometric fluorescent signature to enable unambiguous target identification. Concurrently, we design, build and validate REM-DREAMing, a 4-color, massively-parallel, dHRM platform comprising dual, 400,000 picowell arrays and includes a 4-color imager to decode the 3-color fluorescence signatures and perform HRM methylation analysis (green channel) for each set of 27 targets. Lastly, we assess the clinical sensitivity of the REM-DREAMing assay to detect and identify cancers in a cohort of 100 liquid biopsies from patients with various type and stages of cancers.

### Dual, 27-plex REM-DREAMing Assay Panels Targeting a Panel of 50 Pan-Cancer Detecting and Cancer-Identifying Methylation Biomarkers

In some embodiments, the present disclosure provides all assay elements of the two, 27-plex REM-DREAMing assays to achieve detection and identification of 6 different cancer types. We select the top 50 methylation biomarkers for pan-cancer detection and cancer tissue identification. We leverage the DREAMing primer design software to identify candidate pools of 27 DREAMing primer pairs with minimal primer-dimer interactions, each targeting 25 methylation biomarkers and 2 control loci. Following analytical validation of each REM-DREAMing assay, TaqMan probes are designed for each respective biomarker and will feature a novel, “methylation-agnostic” strategy that will enable hybridization and detection of each target regardless of methylation status/pattern. Unambiguous identification of each target is achieved by using a unique, ratiometric fluorescent detection scheme, whereby each respective biomarker will be assigned 1 of 27 distinct Red:Orange:Yellow signatures, corresponding to the molar ratio of the fluorescently-labeled probes used for its detection. Lastly, the specificity and output fluorescent ratio of each methylation-agnostic probe and corresponding ratiometric encoding, respectively, is validated to ensure adequate performance.

### Selection of Methylation Biomarker Panel

The REM-DREAMing panel includes a total of 50 methylation biomarkers of two types: “pan-cancer-detecting” and phenotype-specific “cancer-identifying.” Identifying the precise tumor phenotype is secondary to accurately detecting the presence of disease in the first place, and to this end we develop and validate primers for all 27 of our cancer-identifying CpG regions that consistently reported high methylation levels in all human cancers as well as minimal methylation in all corresponding healthy tissues (see FIG. 2a) The remainder of the panel is selected from a pool of phenotype-specific methylation patterns (FIG. 2b) to optimize discrimination between the lung, colon, endometrial/ovarian, breast and pancreatic cancer phenotypes that are tested for clinical validation of the REM-DREAMing platform. Lastly, two control loci are included into the panel to normalize DNA loading and calculation of epiallelic fractions, as well as act as for melt temperature calibration throughout the entire microfluidic chip.

### In Silico Design of REM-DREAMing Primer Pools for Multiplex Analysis

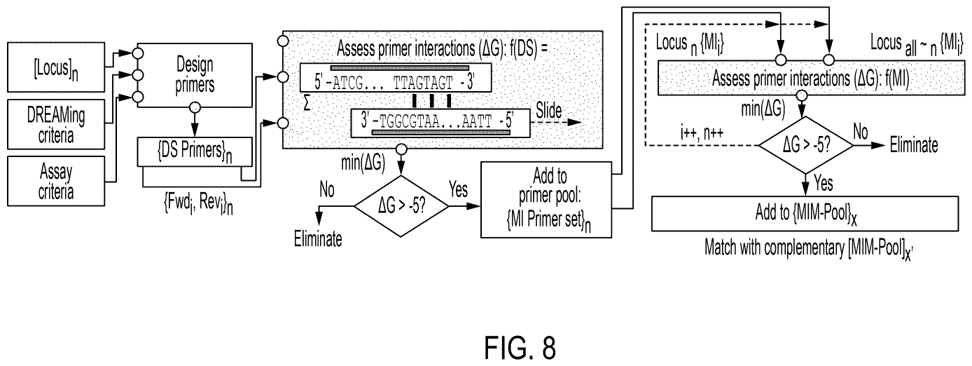

The development of REM-DREAMing assays needs attention to primer design in order to minimize interactions that might result in the formation of primer dimers or otherwise compromise performance. To address this issue, we developed an automated in silico design tool (FIG. 8) that greatly streamlines identification of DREAMing primers and subsequent primer pools suitable for multiplex analysis. Specifically, the algorithm first identifies all DREAMing-suitable (DS) primer pairs for each target locus that meet our established DREAMing design criteria, such as the locations and number of primer CpG-sites, as well as desired assay parameters such as number of CpG-sites and amplicon length for optimizing the detection of fragmented DNA. DS primers for each target-locus are then screened first against themselves to create pools of minimally-interacting (MI) primer pairs. This is accomplished by employing a Gibbs free energy thermodynamic model to compute the likelihood and extent of inter- and intra-primer interactions between the 3′ end of each primer against all other candidate primers for a given target. MI primer pairs for each target-locus are then screened against the MI primer pairs for all combinations of 26 other target-loci. Final MI primer pools comprising MI primer pairs for 27 loci are then matched with MI-Pools targeting the other 27 target-loci.

### Primer Design Data

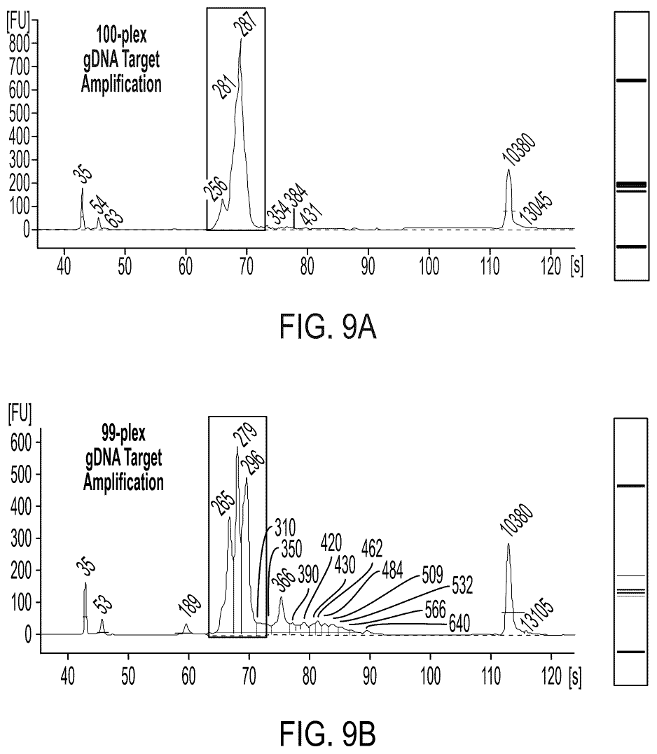

The primer design algorithm for highly-multiplexed targeted bisulfite sequencing is based on similar free energy design principles. Using this method, we design and implement two BS-Seq assays targeting non-overlapping 100-plex and 99-plex methylation biomarkers, respectively. We evaluated the quality of the resulting libraries by standard Bioanalyzer analysis and found the libraries to exhibit little to no detectable formation of primer dimers (FIG. 9).

### Analytical Validation

All MI-primer pools identified above are screened by qPCR in no-template DREAMing assays to check for possible primer dimer formation. Validated pool sets exhibiting no amplification prior to cycle 40 are selected for further evaluation. In pool sets exhibiting primer-dimer products, problematic primer pairs are identified by process of elimination and redesigned, as needed. Subsequently, all primer pairs from non-interacting pools are then systematically evaluated according to two DREAMing primary criteria: difference in cycle-threshold (Ct) values and ability to produce clearly distinguishable single-peak melting temperature differences in unmethylated vs. methylated controls. Assay parameters such as annealing temperature and buffer formulations are optimized further, if necessary. Lastly, synthetic DNA targets are spiked into BSC unmethylated genomic DNA (Qiagen) to verify single-copy sensitivity and 1 in 10,000 specificity (or better) for each assay. The matched MI-Pool pair demonstrating the highest analytical performance for all targets are selected for implementation of the REM-DREAMing assay.

### Methylation Agnostic Ratiometric Fluorescent Probes

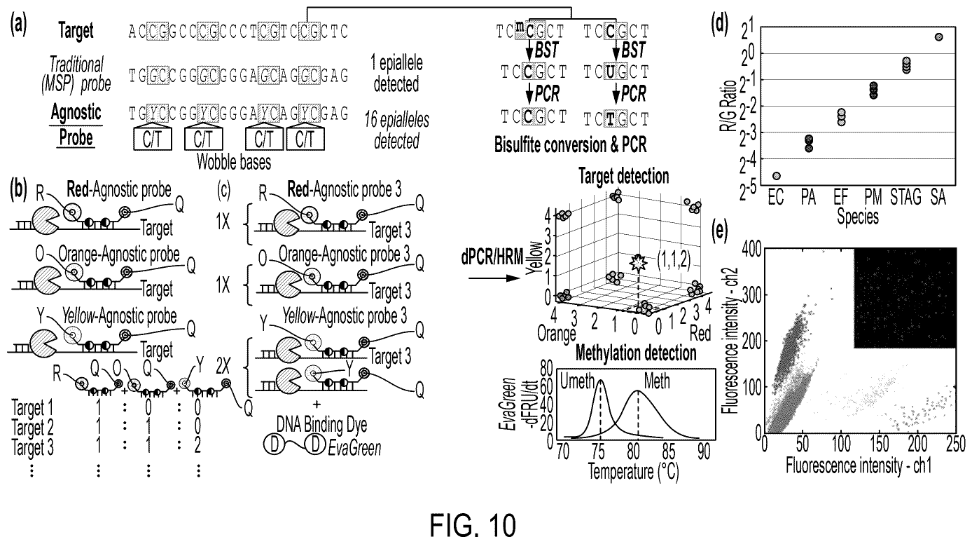

The most notable hurdle that has plagued both DREAMing and other digital-based assays is their limited multiplexing capability. This issue is somewhat exacerbated in methylation assays as DNA probe technologies that might otherwise resolve this issue typically require a priori knowledge of the precise target sequence. Specifically, the bisulfite DNA conversion process leads to the scenario that a single bisulfite converted-epiallelic target produces up to 2n sequence permutations, where n is the number of CpG-sites in target epiallele (FIG. 10a). To circumvent this issue, we use a methylation independent probe design that incorporates so-called “wobble bases” at each methylation-dependent CpG-site in the probe sequence. These wobble bases are created by adding equimolar mixtures of cytosine/thymine (or guanine/adenine) at each CpG site during probe synthesis, yielding an equimolar mixture of 2m probe sequences, where m=number of CpG sites in the probe region. The resulting probe solution is thus “agnostic” to target epiallele methylation status and hybridizes to the target amplicon regardless of template methylation pattern. Higher multiplexing capability is achieved by utilizing three probes for each target. The probes have identical sequences but are differentially labeled with red (R), orange (O) or yellow (Y) fluorophores and quenchers (FIG. 10b). The probes can then be mixed at predefined molar ratios to create a specific fluorescence signature for each target locus. Multiplexing can thus be achieved by assigning a unique, three-color combination to each of the 27 targets per array in our panel. If the multiplexed REM-DREAMing assay is then performed in a microfluidic array chip with absolute digitization, each amplified target can be identified by its respective fluorescence signature ratio, while subsequent melt analysis via DNA binding dyes (EvaGreen) can still be used to measure the melt temperature that directly corresponds to the methylation status of the template epiallele (FIG. 10c).

### Digital Ratiometric Fluorescence Data

We previously developed a similar, 2-color ratiometric fluorescence coding scheme for multiplexed detection of 6 common causative bacteria in urinary tract infections (UTI), including E. coli (EC), P. mirabilis (PM), P. aeruginosa (PA), S. aureus (SA), S. agalactiae (STAG), and E. faecalis (EF) (FIG. 10 d). More recently, we are able to successfully implement this coding scheme into the same digital device used for μ-DREAMing (FIG. 10e).

### Methylation Agnostic Probe Design and Validation

Methylation agnostic probes are designed for each target within the top-performing, matched MI-pool pair identified above. Specifically, TaqMan probes are designed within the intra-primeric region of each target locus using degenerate bases (cytosine/thymine or guanine/adenine), depending on target strand at each CpG-site. Meanwhile, we use prototypes of the REM-DREAMing device to identify the 27 R:O:Y fluorophore ratios of red (ROX, TEX 615, LC Red 640), orange (Cy3, TAMRA, Tye563) and yellow (HEX, JOE) with corresponding quenchers (Black Hole, Iowa Black, TAMRA) and filter set combinations to identify those that yield the highest signal:noise ratio in our microfluidic system. The performance of each target-probe is individually validated for use within each of the MI-Pools and ill performing probe sequences is modified as necessary. Validated probes for each target within the MI-Pool are combined and the analytical performance for each target-assay is reverified. Each target-probe is assigned 1 of 27 most-distinguishable fluorometric ratios and corresponding fluorophore labeled probes synthesized accordingly. Lastly, each methylation-agnostic probe and corresponding ratiometric encoding is validated for specificity and [non-overlapping] fluorescent signal:noise, respectively.

### Design, Fabricate and Validate a Dual 400 k-Well, 4-Color Fluorescence-Decoding dHRM Platform to Perform Parallelized REM-DREAMing for Simultaneous Detection and Identification of 50 Methylation Biomarkers

### Overview

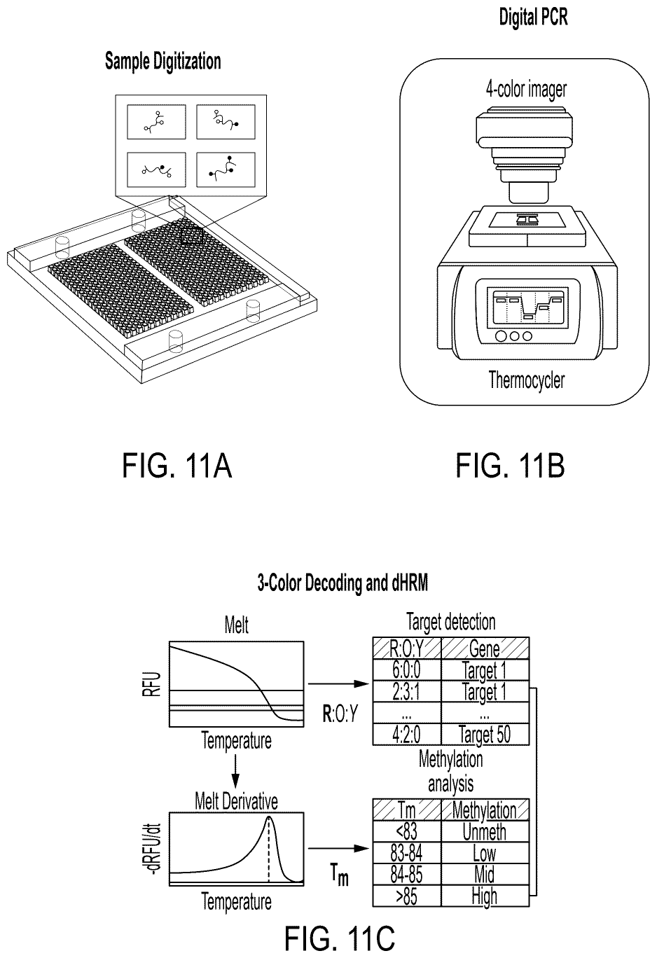

The DREAMing technique was originally demonstrated using only a 96-well microtiter plate and standard qPCR plate reader. This format limits both the throughput and dynamic range of the assay while also requiring significant reagent use/costs to achieve digitization. The small number of wells into which each sample is divided also limits the analytical sensitivity of the digital assay. To address this issue, in some embodiments, the present disclosure provides a microfluidic device and single-color thermal-optical platform that can acquire high-resolution melt curves from 4,000+ nanoliter wells in parallel. This demonstration confirmed that microfluidic parallelization of the DREAMing assay overcomes the limitation of small dynamic range while still able to provide high-quality melt information for interrogation of specific CpG-by-CpG analysis of the amplicons. To develop a highly parallelized multiplex dHRM microfluidic platform, we fabricate a dual-module microfluidic device with 400,000 wells per module, and a four-color real-time imaging platform (FIG. 11). The device contains two modules, each containing 400 k 100-pL wells in which to perform 25-plex DREAMing assays, allowing for a large dynamic range. We previously demonstrated that for a single locus, 4,000 wells were needed to achieve 1:2 million sensitivity and a dynamic range of 5 orders of magnitude. To achieve the same result for a 25-plex assay would require 100,000 wells. Thus, the expansion to 400 k greatly expands the dynamic range of the platform. To enable 25-plex identification, we develop a 4-color imaging platform for post-PCR ratiometric fluorescence analysis. The EvaGreen/FAM channel is used for HRM acquisition, while the 3 other channels: HEX, ROX, and Cy5, are used for ratiometric encoding. We also develop user-friendly, automated software tools to perform the analysis.

### Device Design and Fabrication

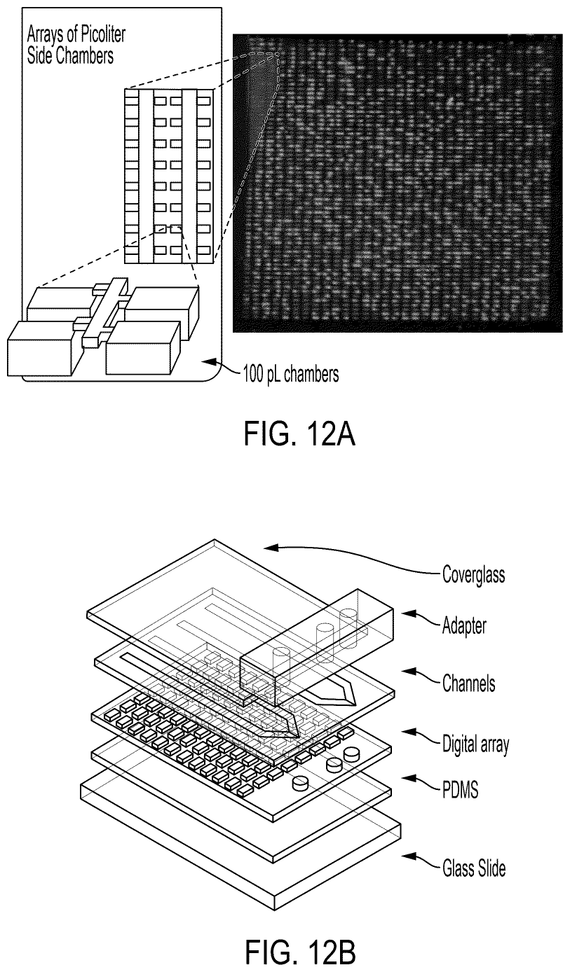

The present disclosure provides a microfluidic array device that performs highly-parallel digital PCR and high resolution melt (HRM) in pL-sized microchambers (FIG. 12). In some embodiments, the material for the microfluidic array chip is fabricated using soft lithography techniques and comprise four distinct layers: a coverglass, a polydimethylsiloxane (PDMS) microchamber layer, a PDMS fluidic channel layer, and a second cover glass. The multilayer structure is designed to tackle the oft-cited problem of evaporation that complicates PDMS-based devices during thermal cycling. Thin PDMS fluidic layers of 30-50 μm are used, as opposed to a commonly used thickness of a few millimeters, to minimize the amount of vapor absorbed by the PDMS. The elevated channel layer is designed to solve the well-known challenge of air or microbubble inhibition of high temperature (PCR) reactions in microfluidic devices. The implementation of a low-aspect ratio elevated channel layer obviates the need for excess reagents in the channels by facilitating evacuation of air from the device during partitioning. We have demonstrated that this multilayer design provides an ~8-fold increase in loading efficiency, from 12% to 80%. We also optimize the volumes of the microchambers, as well as dimensions for the microchannels and pitches, so that the array chip can house more than a half million pL-sized microchambers in a 50 mm×36 mm area. The lower limit of the digital chamber dimensions is dictated by the sensitivity of the optical detection instrument. To ensure achievement of strong fluorescent signals from PCR, we also optimize the conditions of dPCR by adjusting the concentrations of our polymerase, primers, probes, EvaGreen, additives (BSA and Tween20), as well as the annealing temperature. We minimize the separation between digital chambers until adjacent chambers to match the resolution of the optical detection instrument.

### Optical-Thermal Module

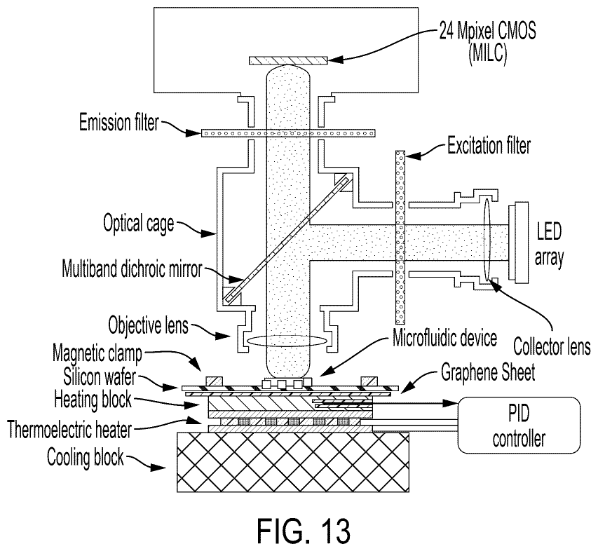

We develop an optical-thermal module that contains a real-time imager and a high-resolution thermal cycler. The imager is constructed using an optical cage system that applies epifluorescence (FIG. 13). An LED array is used as a broad illumination source and a 24-megapixel full-frame CMOS MIL camera (Sony a7, 36 mm×24 mm) is used as a fluorescence detector to image the entire array chip of dimension 54 mm×36 mm. Four filter sliders host 4 exciter-emitter sets that are compatible with the fluorophores of EvaGreen and the yellow, orange and red dyes for the probe labeling. The thermal cycler is built based on thermoelectric heating/cooling module by modifying the prototype previously developed. Temperature is controlled through an array of independent thermoelectric elements, a metal heating block, and heat spreading layers of graphene and silicon to achieve precise thermal uniformity over the entire chip area. We add magnetic clamps to ensure robust and uniform thermal contact of the microfluidic device to the heating surface.

### Image Acquisition and Melt Curve Analysis

Software specific to these implementations is needed to analyze the melt profiles of up to a half-million reactions. We implement algorithms for automated segmentation of reaction wells and normalization of chamber-to-chamber signal variations caused by non-uniform heating and illumination across the field of view. During melt curve analysis, the temperature is ramped at 0.05° C./s. At each temperature step, an image containing all digital reaction chambers is acquired. A program extracts the fluorescent signals from the image, to construct and process the melt curves. Calibration and controls for both digitization and HRM analysis are achieved by incorporating discrete copy numbers of synthetic oligonucleotides for each target locus into the assay Mastermix. The sequence of the synthetic oligonucleotides contain only the primer and corresponding TaqMan-probe targets, to yield low-temperature melt curves easily identifiable by HRM analysis. The melt temperatures (Tm's) of the synthetic DNA amplicons for each selected target also act as a reference for correction of non-uniform temperature profiles across the array. The raw melt curves will be smoothed by a Savitzky-Golay smoothing filter with user-defined parameters, followed by interpolation with temperature adjustment such that the Tm of the calibrator strand is aligned across profiles obtained from all chambers. After calibration of chamber-to-chamber variations (for both optical signals and temperature) and curve smoothing, melt curves are normalized to decouple the contribution of the temperature-dependent background fluorescence change from the true fluorescent signals associated with DNA melting. The feasibility of the proposed programs for imaging and melt curve analysis has been demonstrated by performing digital HRM on the aforementioned prototype digital chip containing 4,096 chambers (see FIG. 5). Based on this development, the general design principles of this module are retained and expanded to include optimizations in individual parts primarily to further improve the fluorescence signal of digital HRM and to reduce assay time to completion through the integration of the microfluidic devices.

### EXAMPLE

### Example 1: Ultrasensitive Epiallelic Profiling of DNA Derived From Routinely-Collected Pap Specimens for Detection of Ovarian Cancer

### Introduction

High-grade serous ovarian cancer (HGSC) remains the most lethal gynecologic cancer and the fifth most common cause of cancer-related death for women in the United States. Over 22,000 women in the U.S. are diagnosed with ovarian cancer annually and approximately 14,200 (>60%) women die each year from this disease. The poor survival of all HGSC patients is largely attributable to delayed diagnosis, as approximately 75% of patients do not present symptoms until an advanced stage when curative resection is no longer possible. Indeed, various epidemiological studies have found that women whose ovarian cancer is diagnosed at an early stage have significantly higher 5-year survival rates (>85%) as compared to those diagnosed at late stages (<30%).

The drastic improvement in survival among ovarian cancer patients diagnosed at early stages of disease has engendered considerable effort over the past several decades to develop tests for screening and early detection of HGSC. Most notably, a number of large-scale screening trials based on protein biomarkers, such as cancer antigen 125 (CA125), and/or imaging approaches, such as transvaginal ultrasound (TVU), have been performed; however, these approaches have yet to demonstrate meaningful survival benefit. Consequently, several national organizations, including the US Preventative Services Task Force, do not currently recommend routine screening for ovarian cancer due to the observation that “the potential harms outweigh the potential benefits”. There thus remains an urgent need for the development of novel, minimally-invasive diagnostic approaches that are not only able to reliably detect HGSC, but to do so at sufficiently early stages to improve patient outcomes.

Numerous studies have now provided abundant evidence that the majority of HGSCs begin as precursor lesions in the fallopian tube. These lesions are thought to shed precancerous cells that spread to the ovaries, where they rapidly evolve to form ovarian tumors. However, due to its anatomical location, the upper female reproductive tract, including the ovaries and the fallopian tubes, is difficult to screen noninvasively. Thus, the vast majority of these molecular diagnostic assays have relied upon blood-based liquid biopsies as biospecimens for detection.

Recent work has suggested that cells derived from ovarian tumors and/or precursor lesions are able to freely pass from the fallopian tubes into the cervical-vaginal fluid, which is already collected during a routine gynecologic examination. This has inspired a new approach to gynecological cancer diagnostics that employs next-generation sequencing (NGS) to detect cancer-associated mutations within routinely-collected Pap specimens. While these technical platforms, called “PapGene” and “PapSEEK”, have demonstrated excellent ability to detect endometrial cancers (ECs), the detection of HGSC has proven significantly more challenging. One reason for this discrepancy is that cells from HGSC tumors are often present in Pap specimens only at low fractional concentrations (<0.1%) within a large population of healthy cells. This poses particular challenges for sequencing-based techniques as, even with the incorporation of so-called unique molecular identifiers (UMIs), NGS platforms still generally struggle to detect variant fractions below 0.1%.

In addition to genetic abnormalities, studies by us and others have demonstrated that virtually all ovarian cancers exhibit aberrant DNA methylation. Biomarkers based on cancer-specific DNA methylation are a particularly attractive option for the early detection of cancer. First, methylation is a conspicuously early event in carcinogenesis, often arising even prior to tumor development. Recently, we were the first group to directly confirm this phenomenon in ovarian carcinogenesis by using genome-wide methylation analyses to identify a limited set of genomic loci that are consistently and specifically hypermethylated at all stages of HGSC including early-stage precursor (STIC) lesions. Secondly, DNA hypermethylation is inherently chemically and biologically stable and, unlike point mutations, often occurs in CpG islands that span hundreds of basepairs and can thereby be collectively probed to greatly enhance overall “signal” to improve performance and detection limits. Third, cancer-specific hypermethylation typically exists in hundreds to thousands of different regions throughout the cancer genome. This redundancy is particularly advantageous, as panels of methylation biomarkers can be carefully selected to achieve high clinical sensitivity and specificity, as well as to provide robustness against background signals stemming from healthy tissues.

Yet despite these tremendous advantages, the detection of methylation biomarkers in complex samples such as Pap specimens remains challenging. In particular, while aberrant methylation is present in the vast majority of STIC lesions and HGSC tumors, the methylation patterns themselves typically arise progressively and in a stochastic manner, leading to clonal cell populations with random (heterogeneous) methylation patterns and profiles. This poses considerable challenges for commonly-employed techniques, such as methylation-specific PCR (MSP) and digital-MSP (dMSP) that are designed to only detect specific, predefined methylation patterns (typically densely-methylated). This drawback critically undermines the ability of these tests to detect and quantify tumor DNA, particularly at early stages of disease and in challenging samples such as liquid biopsies and Pap specimens. On the other hand, targeted bisulfite NGS remains relatively expensive for routine testing, requiring substantial sample prep costs and time. Furthermore, NGS techniques typically struggle to achieve even 0.1% analytical specificity, particularly in repetitive sequences such as those found within CpG islands. While some recent, well-publicized approaches have sought to circumvent these fundamental limitations through multivariate (e, g., CancerSEEK) and/or extraordinary large biomarker panels (e.g., cfMeDip-seq and the GRAIL project), these methods are too expensive for routine use and rely on complex algorithms that will need to be validated in very large-scale studies (>10K subjects) to prevent overfitting.

To address this challenge, we previously developed an alternative approach called DREAMing that provides a facile and inexpensive means ideally suited to the detection and assessment of ultra-rare epiallelic variants such as those found in liquid biopsies. DREAMing was originally implemented in standard 96-well plates by employing semi-limiting dilution and precise melt curve analysis to distinguish and enumerate individual copies of epiallelic species at single-CpG-site resolution in fractions as low as 0.005% directly from liquid biopsies. We recently implemented DREAMing into a microfluidic platform called HYPERmelt, which is capable of detecting as few as 1 in 2 million copies of heterogeneously methylated DNA. The platform digitizes DNA molecules into thousands of 1-nL chambers, then uses methylation-preferred primers to amplify all possible methylation patterns of a single locus. We then perform parallelized digital high-resolution melt to analyze molecule-by-molecule methylation heterogeneity within a sample. This simple-to-use platform achieves the high-sensitivity, low-cost, and practicality necessary for routine clinical use.

In the present study, we developed a data analysis pipeline that leverages the rich datasets provided by the platform, interrogating the fractional CpG methylation status on a molecule-by-molecules basis for each locus, to determine an overall predictive score for ovarian cancer. The pipeline readily scales from individual biomarkers to panels, and is implemented such that it outputs relevant biological features which may be informative to further studies. We substantially improved upon the throughput of the platform to facilitate screening of clinical sample cohorts, namely through design changes that permit the assessment of larger sample volume (up to 40 μL) of up to 4 samples in parallel. Finally, we sought to apply the improved HYPER-Melt platform to evaluate the methylation patterns of ovarian cancer-specific methylation biomarkers in DNA derived from Pap specimens to determine whether such an approach could lead to improved clinical performance for routine screening applications.

### Platform Overview

Early cancer tissue exhibits highly variable DNA methylation patterns, wherein both the number and location of CpG sites that are methylated within a locus demonstrate stochasticity. Intermediate, variable DNA methylation patterns are exhibited in ovarian cancer cells as well as in precursor lesions, which may migrate to the cervix and be collected via routine Pap exams. The Hypermelt platform utilizes thermodynamic principles to quantify the number of methylated CpGs within a locus on single copies of DNA. The platform performs this analysis on thousands of individual molecules in parallel in order to produce a quantitative output of molecular variability within a sample. This high-throughput parallelization through microfluidic manipulation enables ultrasensitive, low-cost DNA methylation profiling that is amenable to routine screening.

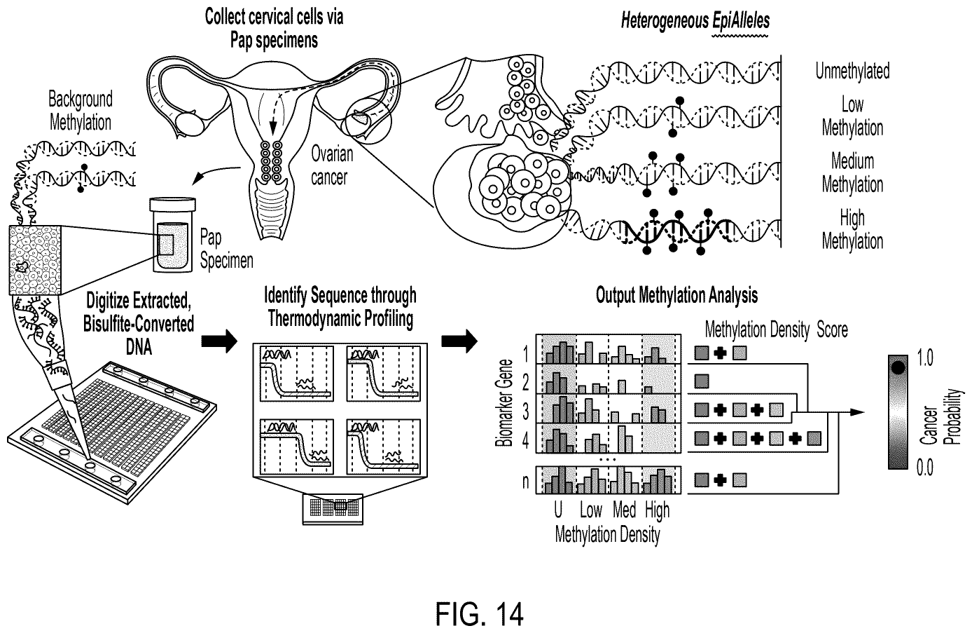

To assess the clinical feasibility of using cancer-specific heterogeneous methylation as an early biomarker for ovarian cancer, we analyzed DNA extracted from routinely-collected Pap specimens on the Hypermelt platform. First, DNA was extracted from Pap specimens and bisulfite-treated to convert unmethylated cytosine to uracil, thereby translating alterations in DNA methylation to changes in primary sequence (FIG. 14). Samples are then loaded onto a microfluidic chip consisting of 4 modules, each containing 10,000 1-nL wells that act to digitize rare target molecules into individual reaction chambers. Highly parallelized digital PCR (dPCR) was performed on the device using methylation-preferred primers to amplify all possible epialleles. A thermal-optical platform was developed to perform digital high-resolution melt (dHRM) analysis to determine the epiallelic identity of each amplicon. HRM analysis leverages bisulfite-induced changes in primary sequence from cytosine to uracil, which decreases the thermodynamic stability of dsDNA, resulting in a measurable shift in the temperature at which a sequence denatures, termed “melt temperature.” Melt curves were used to detect methylation patterns for each locus in the biomarker panel. We introduce a comprehensive analysis of the variable methylation patterns across the panel, and develop an algorithm to combine the multidimensional information and to produce a probability score that the sample contained tumor-derived DNA.

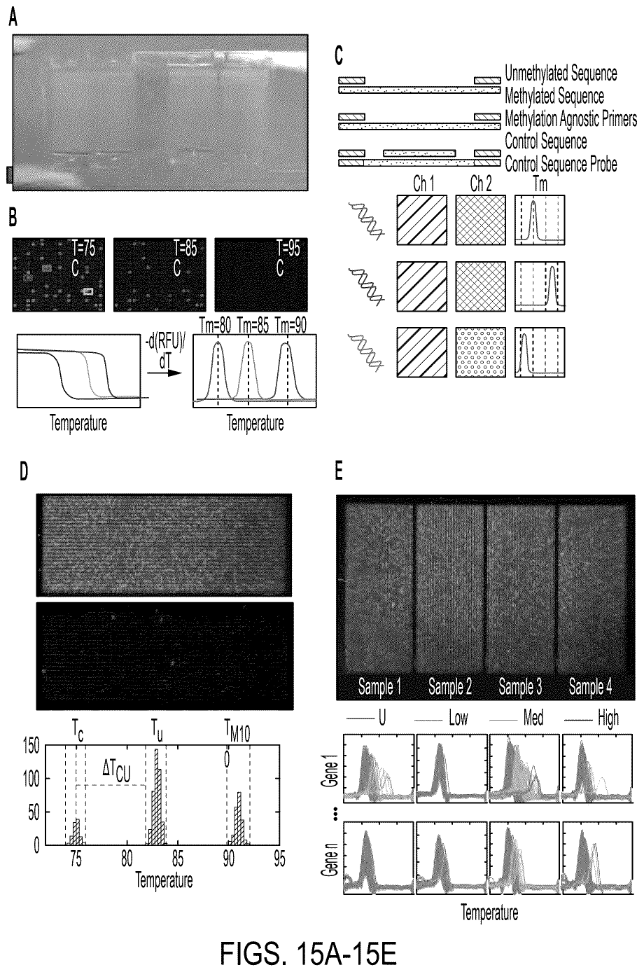

Previously, we described the basic principles for the design of the microfluidic device and thermal optical platform to perform digital HRM. Briefly, we developed an ultra-thin microfabrication technique to produce a microfluidic array that digitizes DNA into 1 nL chambers and prevents evaporation during high-temperature reactions such as PCR. To perform dHRM, we developed a thermal optical platform consisting of a flatbed thermal cycler and wide-field imager that captures fluorescence images of the entire array at each 0.1° C. increment. Significant improvements were made to the microfluidic device and thermal-optical platform to interrogate the methylation patterns of thousands of individual molecules with increased efficiency, sample input volume, and throughput. We designed a microfluidic device containing 4 nanoarray modules to be able to run up to 4 samples in parallel (FIG. 15A). Each module contains 10,000 1-nL size chambers to digitize DNA. The reaction mix is prepared off-chip, and then loaded into the device via vacuum-based loading. Partitioning oil is then pressurized through the channels to digitize the chambers following our previous technique.

The principles for discrimination of methylation patterns based on melt curve analysis have been laid out previously. Briefly, locus-specific methylation-preferred primers enable amplification of all methylation patterns (epialleles). Sequence profiling through dHRM is then accomplished by measuring the thermal stability of the methylation-dependent amplicon sequences by observing the release of a dsDNA binding dye as a function of temperature. Fluorescence images of the entire chip area are captured simultaneously via a wide field-of-view MIL camera during temperature ramping to record the denaturation of each digitized target (FIG. 15B). The fluorescence signal is extracted and filtered to produce a melt curve for each amplicon. The negative derivative of the melt curve with respect to temperature contains a peak, which is termed the “melt temperature” (Tₘ) of the sequence, and correlates to the methylation pattern.

Methylation biomarker candidates suitable for early detection of HGSC were derived from our previously reported characterizations of genome-wide methylation in ovarian tumors and ovarian cancer precursor STIC lesions. To maximize assay specificity in heterogeneous Pap specimens, biomarker loci exhibiting both HGSC- and STIC-specific hypermethylation as well as minimal methylation in healthy gynecological tissues, including fallopian tube, endometrium and cervical mucosae were specifically selected. From these studies, we identified nine loci that met these criteria and also demonstrated high sensitivity and specificity: IRX2, ZNF154, c17orf64, c5orf66, TBX4, miR24, TUBB6, LRRC32, and PCDHG.

Methylation-agnostic primer pairs were designed for 9 candidate loci based on previously-described criteria. In brief, design constraints were as follows: (I) inclusion of 0, 1 or 2 CpG dinucleotide in each primer toward the 5′ end; (II) primer melting temperature near 60° C. and within 2° C. of each other; (III) no significant hairpin or primer dimer formation. All primers were ordered from Integrated DNA Technologies (IDT). Primer pairs were evaluated through the analysis of cycle thresholds (Ct) values and melt curve profiles based on two major criteria: (1) Ct values of primer dimer is equal to or later than 40; (2) the ability to produce clearly distinguishable single-peak melting temperature differences in unmethylated versus methylated controls. Analytical sensitivity was further evaluated through serial dilution of bisulfite converted methylated control DNA in negative-control converted unmethylated genomic DNA.

After initial analytical validation, each DREAMing assay was calibrated on the microfluidic device to establish the correlation between Tₘ and epiallelic methylation density. This was achieved using synthetic sequences equivalent to the bisulfite-converted, fully methylated epialleles for each of our target loci, while bisulfite-treated (BST) genomic DNA from healthy women was confirmed to be unmethylated at the target loci and used as negative controls.

To improve confidence in the digital readout and mitigate any run-to-run variation, we additionally designed and implemented an internal calibration method using unique control sequences to quantitatively validate digital amplification efficiency and adjust for thermal variance within and between all modules in each experiment. Unique synthetic control sequences were designed specifically for each locus (FIG. 15C). The 5′ and 3′ ends of each sequence are complementary to the methylation-preferred primers. The internal sequence was designed to be complementary to a ROX probe. The probe sequence was the same for all loci to melt at a temperature lower than the unmethylated DNA for each locus. A discrete number of control sequences were spiked into each reaction mix before loading into a module of the device to be digitized in discrete wells throughout the module. Digital wells containing sample DNA with the target of interest are amplified and detected via the DNA binding dye, whereas wells containing the control sequences exhibit fluorescence from both the intercalating dye channel and separate probe channel to distinguish them from amplified sample templates. The Tₘ of the control sequences could then be used to establish accurate calibration of the melt temperature to methylation density pattern. For both the unmethylated and methylated populations, the differences between the control sequence Tₘ (ΔT_{CU} and ΔT_{CM100}) were calculated respectively. This produces a linear calibration curve to establish the conversion between Tm and methylation density per locus to be used for sample data collection.

### Epigenetic Profiling of DNA From Pap Specimens

We next sought to assess the diagnostic potential of the nine biomarker-assays by comparing methylation heterogeneity at the corresponding target loci in DNA derived from Pap specimens collected from healthy women during routine gynecological examinations in comparison to those from women diagnosed with ovarian cancer. The initial cohort included 20 liquid-based endocervical brush samples from women with advanced stage (FIGO stage III and IV) ovarian serous carcinomas and 23 samples from cancer-free women, totally to 387 digital assays. Those specimens were obtained from women before gynecologic surgeries following the institutional research protocol. DNA was extracted from cell pellets within the Pap specimens and underwent bisulfite conversion for analysis on the platform.

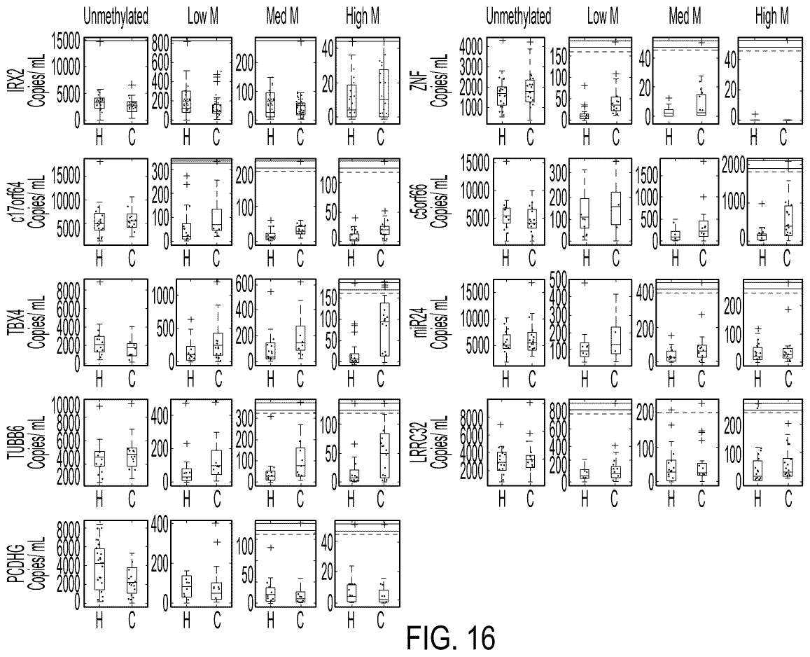

For each locus-assay, a reaction mixture containing 5 μL of BST-DNA from each Pap specimen was loaded into one of the four microfluidic modules on the PapDREAM device. Following on-chip PCR, HRM was performed by gradually increasing temperature to denature the target amplicons while monitoring fluorescence. A Matlab script extracted the fluorescence for each amplicon to produce melt curves. The negative derivative of the fluorescence with respect to temperature was taken and the location of the peak defined as the Tm. Methylation levels were then calculated for each amplicon based on the calibration curve for each locus. The detected epialleles for each locus and sample were first stratified into unmethylated molecules, low methylation, medium methylation, and high methylation based on the melt temperature of the amplicons. The number of molecules within each methylation stratification was then normalized to the volume of input DNA into the reaction. (FIG. 16)

One key advantage of the HyperMelt system used for the PapDREAM method is the ability to rapidly quantify heterogeneous methylation of individual template molecules. This capability ostensibly provides improved sensitivity for detection, particularly at early stages of disease when methylation patterns are expected to be more variable and less dense than in later stage disease. Our recent findings detected highly heterogeneous methylation in numerous individual loci within fresh-frozen HGSOC gynecological tissue and minimal methylation within healthy mucosal tissues. Indeed, incorporation of methylation density analysis has been shown to significantly improve clinical performance of methylation biomarkers, but has yet to be extensively utilized due to the challenges of using existing tools to measure intermediate methylation within challenging samples with low DNA levels. Thus we sought to determine if the presence and prevalence of intermediately methylated molecules within a locus could provide further insights.

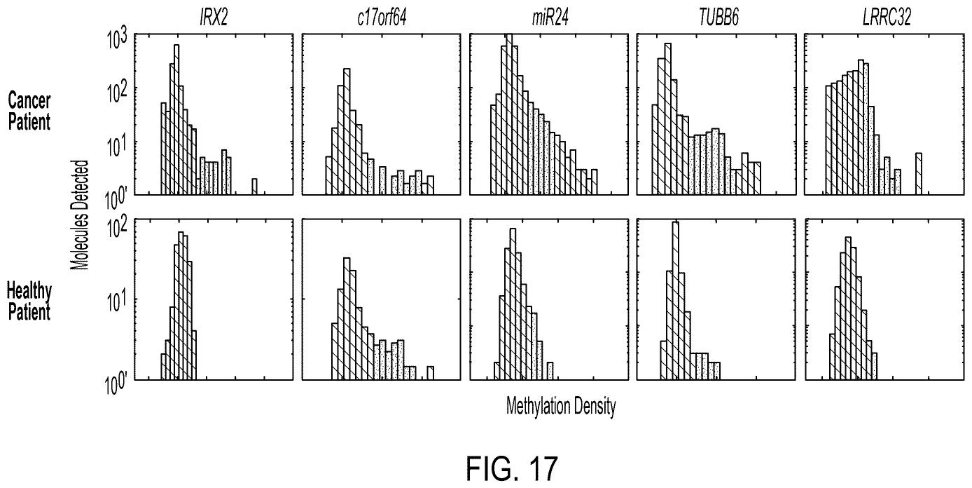

We developed a quantitative single-molecule methylation density analysis method by first categorizing each detected epiallele according to its methylation density into 10 percentile bins (0%, 10%, 20%, . . . 100%) to create a methylation density histogram for each respective target-locus (FIG. 17). Each bin was then used as a threshold for counting the number of epiallelic molecules present per sample per locus, wherein a 40% methylation threshold compares the number of molecules that exhibited methylation patterns of at least 40%, up to 100%. Furthermore, we sought to investigate the prevalence of molecules containing altered DNA within varying levels of background healthy molecules. The relative significance of the fraction of epigenetically altered DNA compared to the concentration of altered DNA is not well characterized within most liquid biopsy applications, as existing technologies are designed for sensitive detection of methylated molecules at the expense of quantification of other methylation patterns. The simultaneous quantification of methylated and unmethylated epialleles within this platform allows the measurement of the fraction of epialleles present in a sample. Since cell-free DNA levels as well as cellular levels per pap specimen vary greatly from person to person, we sought to determine these fractional values by also calculating the epiallelic fraction of each locus present in each sample.

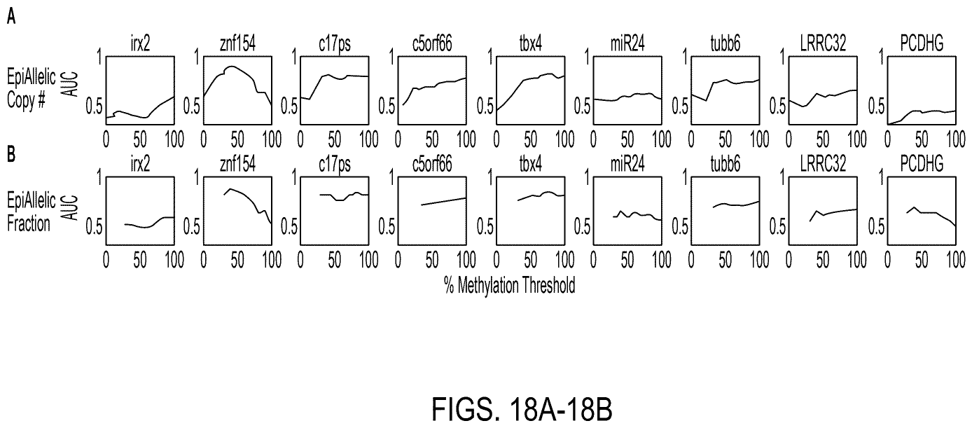

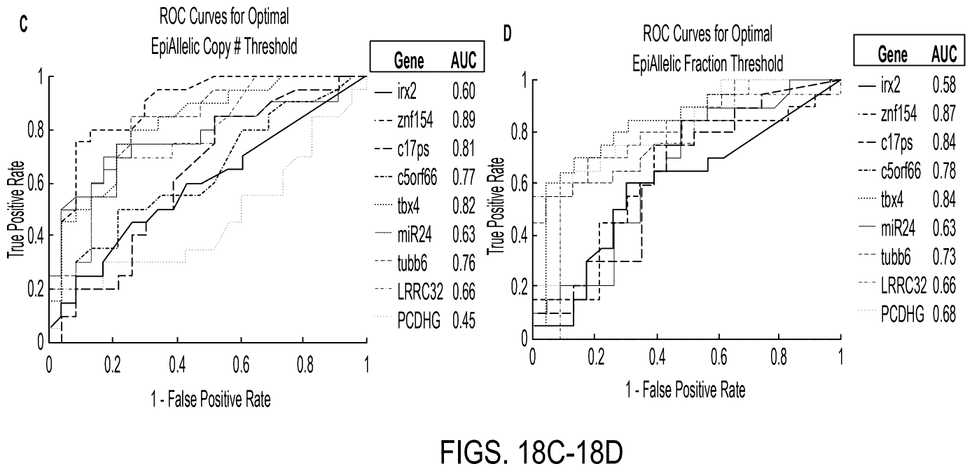

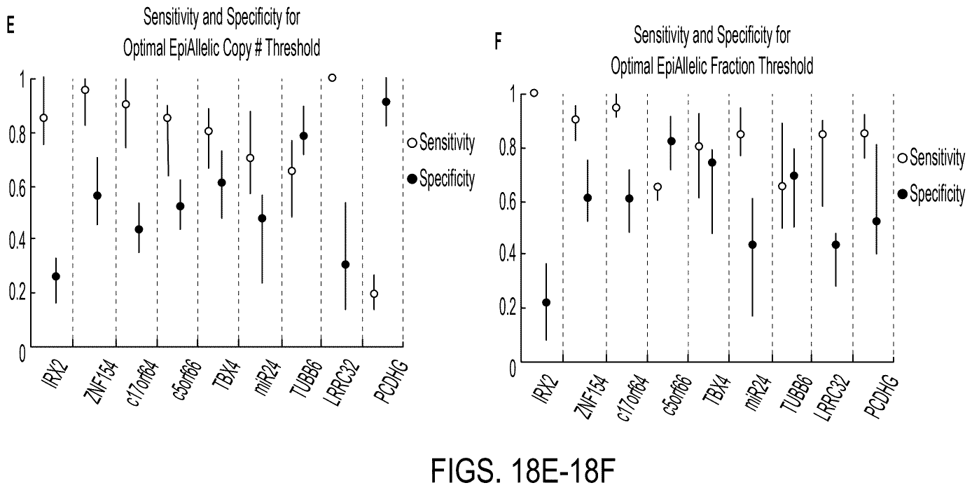

The area under the receiver-operating-characteristic (ROC) curve (AUC) for the training cohort was then calculated according to the methylation density threshold applied to each locus. We then compared how each methylation density threshold affects the AUC for each individual locus (FIG. 18A). We observed that for 3/9 loci, the fully methylated epiallelic copy number resulted in the highest AUC. For 3/9 loci and 2/9 loci, a threshold around 40% and 80% methylation respectively performed similarly or slightly better than a 100% threshold. Notably, the optimal methylation threshold for ZNF154 was determined to be 40% and significantly declined as the threshold was increased, a finding that was qualitatively consistent with a prior study exploring methylation density thresholding for ZNF154 for blood-based detection of HGSC.

To explore how normalizing the epiallelic fractions to the total number of epialleles in each sample might affect clinical performance, we performed a similar data analysis to identify optimal epiallelic fraction cutoffs for each locus-assay (FIG. 18B). The results of this analysis indicated that the effect of density thresholding and optimal AUC values were similar using either the absolute count of hypermethylated epialleles or epiallelic fraction.

Once the optimal methylation density thresholds were determined, we compared the ROC performance of the individual DREAMing assays (FIG. 18C, D). Using epiallelic copy number, ZNF154 performed the best with an AUC of 0.89, followed by TBX4, c17orf64, c5orf66 and TUBB6 (0.82, 0.81, 0.77, 0.76). Similarly, ZNF154 performed the best using the epiallelic fraction method with an AUC of 0.87, followed by TBX4, c17orf64, c5orf66 and TUBB6 (0.84, 0.84, 0.78, 0.73). We also compared the sensitivity and specificity of each locus using the two methods (FIG. 18E, F). Though the overall AUC values were similar between the two methods, clinical specificity was notably higher using the epiallelic fraction values than the absolute copy numbers for most loci. Overall, these results indicate that while methylation density thresholding can often improve biomarker performance, each respective threshold value must be determined empirically and will likely be dependent upon the particular biomarker, biospecimen type and intended application.

### Methylation Biomarker Panel Performance

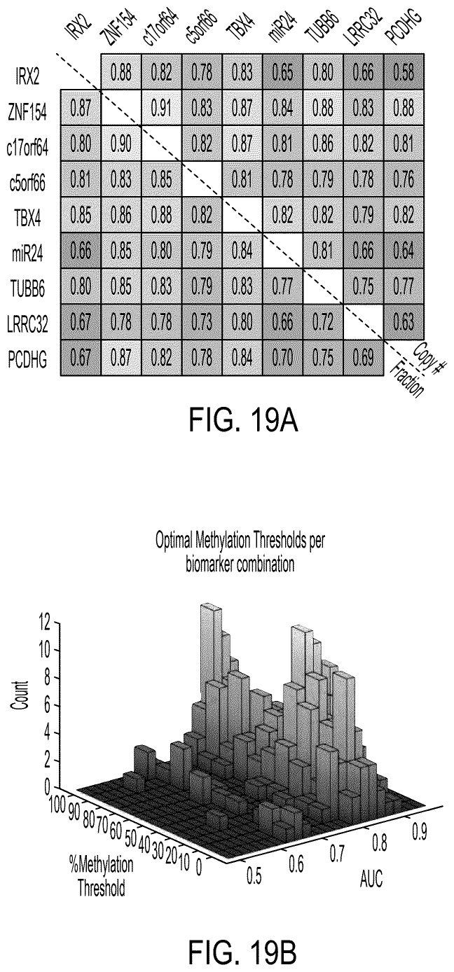

Next, we analyzed the performance of the biomarker panel in combination. For each set of loci, we determined the optimal methylation threshold for each marker for each specific combination. Methylation data above each threshold was then combined into a single score. Receiver-Operator Characteristics (ROC) and corresponding area under the curve (AUC) for each combination was calculated. Here we present the AUCs for the top combination of any 2 biomarkers in the panel (FIG. 19A). Multigene analysis using only two biomarkers can offer improvements in biomarker accuracy, improving the top AUC to 0.91. We observed that combinations using epiallelic fractions performed slightly better than epiallelic copy number.

Next we sought to analyze the significance of various intermediate methylation patterns on the performance of biomarker panels. We compared the methylation density thresholds levels that produced the optimal AUC for each single and multigene combination of biomarkers (FIG. 19B). Once again, the optimal methylation density thresholds were locus dependent and ranged from intermediate methylation (e.g, ZNF154 40%, TUBB6 50%) to full methylation (e.g. IRX2 100%, c5orf666 100%). By comparing the frequency distribution of the methylation thresholds providing the top AUCs per combination, we see two higher-frequency clusters of methylation patterns in the 30-50% range and 80-100% range. We believe this result suggests that the effect of intermediate methylation in cancer development warrants further investigation.

### Validation in Pap Specimens From Women With Early-Stage HGSC

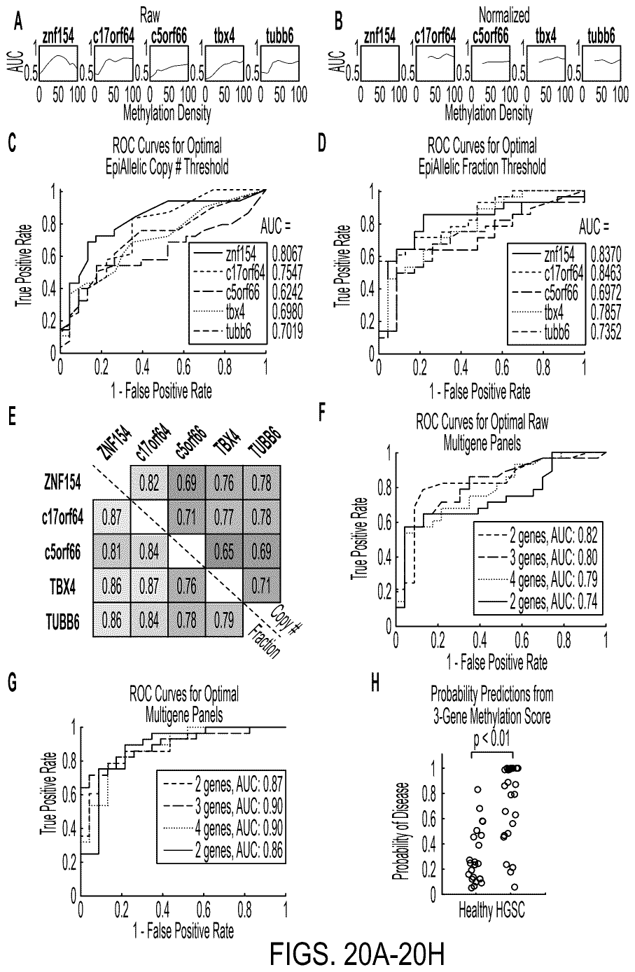

From this cohort of healthy and late stage HGSC specimens, we identified the top 5 potential biomarkers, ZNF154, c17orf64, c5orf66, TBX4, and TUBB6, for further investigation with Pap specimens collected from early (stage 1) HGSC patients. We detected methylation patterns for these loci for 8 early-stage patients on the microfluidic platform. Next, we calculated the optimal methylation density threshold for each individual locus using the 23 healthy samples and all 28 cancer samples (20 late+8 early). We repeated this analysis for both epiallelic copy number and epiallelic fraction (FIG. 20A, B).

The optimal methylation thresholds for each individual locus were similar when compared to only late stage results. The epiallelic fraction method resulted in higher AUCs overall for each locus compared to the epiallelic copy number (FIG. 20C, D). For any individual biomarker, the performance when including the early-stage samples using epiallelic fraction is similar to that of only late stage analysis.

We again investigated the combination of these markers into a single-score multigene panel. Optimal methylation thresholds were determined for each locus in a combination ranging from 2 to 5 genes. Methylation levels above these thresholds were then added together to produce an overall score. Receiver-Operator Characteristics (ROC) and corresponding area under the curve (AUC) for each combination was calculated. The performance of just 2 of these genes in combination results in an AUC of 0.87 (FIG. 20E). ROC curves for the optimal combination of 2-5 genes are shown (FIG. 20FG). Using this simple thresholding method with only 3 biomarkers, an AUC of 0.9 (specificity 100%, sensitivity 64%) is achieved that includes early stage ovarian cancer specimens.

To provide a probability of an individual having HGSC, we fit a logistic regression model to the overall three-gene panel methylation density score and calculated predicted probabilities of disease for each patient using leave-one-out cross validation. The results show that cancer patients are significantly more likely to have a methylation density score resulting in a higher probability of cancer than healthy patients (FIG. 20H).

There are a number of drawbacks to this method that warrant discussion. The current implementation of the presented assays as a single locus per module limits the throughput towards detection of larger patient cohorts or larger biomarker panels. The method is readily extendible for simultaneous, multiplex biomarker interrogation by carefully tuning assay parameters such as primer design and annealing temperatures for future cohorts. However, interrogation of biomarker panels larger than ~five may require more complex multiplexing methods, and thus other techniques may be more suitable for preliminary biomarker discovery. Nevertheless, these results and others suggest that even a limited number of biomarkers (1-4) may be clinically informative, and additional loci may not substantially improve diagnostic accuracy. Possibly, the diagnostic accuracy of this technique may be limited by the tumor phenotype. Further studies will be needed to determine what fraction of HGSCs exhibit behavior resulting in anomalous cells appearing in the endocervical canal, and thus amenable to detection via Pap specimens. Finally, though these early results are promising, further studies with a larger patient cohort should be performed to validate the findings.

There are several advantages to our novel PapDREAMing approach for ovarian cancer screening. First, the single-molecule sensitivity even among high healthy background is critical for early detection, as mutant allele frequencies of ovarian cancer in pap specimens have been shown to be <1.0% and lower. Utilization of pap specimens minimizes barriers for integration into the clinical workflow by leveraging an already routine practice. However, such a strategy necessitates extremely sensitive detection methods to detect rare cancer-specific epialleles. Current practice for evaluation of pap specimens for cervical cancer recommends analysis of a minimum of 15,000 cells. DNA from ovarian cancer or precursor tissue is expected to come from cellular debris or “sloughed-off cells” and fragments that travel from the fallopian tube into the endocervical canal. Thus, the low prevalence of 1 aberrantly methylated epiallele in 15,000 (0.0067%) or lower can be expected for early disease. Whereas most other technologies struggle to detect variants below 0.1%, this demonstrated sensitivity of this platform to detect variants as rare as 0.00005% can readily overcome this challenge. Furthermore, such results can be achieved via a low-cost and practical workflow that is amenable to routine screening. Finally, this simple and intuitive analytical method can be validated without complex computational analysis tools, and may provide insight into biological mechanisms underlying disease progression.

### Methods

### Device Fabrication

Devices were fabricated using standard photolithography and ultra-thin soft lithography techniques. To fabricate the reusable master mold, a silicon wafer was dehydrated and oxygen plasma treated (Technics PE-IIA) at 80 watts for 1 minute. SU-8 3050 photoresist (Microchem) was spun on the wafer at 2600 rpm for 1 minute. Then wafer was baked at 95° C. for 20 minutes, then exposed in the mask-aligner at 300 J/cm², developed, and baked at 200° C. for 1 hour. Microfluidic chips were fabricated using soft lithography and our own ultra-thin layering technique. A 15:1 mixture of poly dimethyl siloxane (PDMS, Ellsworth) was spun on the silanized pattern wafer at 500 rpm, while a 6:1 mixture of PDMS was spun on a blank wafer. Both wafers were baked for 6 minutes at 80° C. The sacrificial layer was then removed, overlaid on the pattern layer, and baked briefly again. A blank glass slide (Ted Pella) was cleaned by rinsing with water and dried. PDMS (15:1) was spun on the glass slide at 2100 rpm; then baked at 80° C. for 6 minutes. The two PDMS layers on the pattern wafer were removed jointly. Both the pattern PDMS layer and the blank PDMS on the glass slide were both oxygen plasma treated at 40 W for 45 seconds. After bonding, the sacrificial PDMS layer was removed from the pattern. Finally, a tubing adapter and glass coverslip were oxygen plasma bonded to the top surface of the chip. The device was then baked at 80° C. overnight, sealed with a piece of thin adhesive tape over the inlet and outlet, and desiccated for a minimum of two hours before use.

### Clinical Sample Collection and Preparation

Endocervical brushed cells were collected in the ThinPrep® Pap Test reagent and kept at room temperature until use as previously described (PMID: 23303603, 29563323). DNA was extracted from the collected samples using a Qiagen DNA purification kit from cell pellets. The DNA amount was quantified using a PCR method and subject to bisulfite conversion for the assay. The diagnoses of ovarian cancer were confirmed by corresponding pathology reports.

### Sample Preparation

A simple diagram summarizing sample preparation procedure is shown below (Figure S1). DNA extraction from Pap specimens was performed with QIAamp DNA FFPE Tissue Kit (QIAGEN) according to manufacturer's protocol. Briefly, cells from 1 mL of Pap specimen were digested with proteinase K for 1 hour at 55-60° C. DNA was transferred to QIAamp MinElute columns placed in a 2 mL collection tube, washed by washing buffer and ethanol, and consequently eluted into 30 μL of elution buffer. Long interspersed nuclear element 1 (LINE-1) standards were used to estimate the overall cfDNA copy numbers with 300 nM of forward primer, 5′-AGG GTT TTT ATG GTT TTA GGT T-3′, 300 nM of reverse primer, 5′-ATC CCT TCC TTA CAC C-3′, spanning 82-bp regions, and 100 nM of probe, 5′-\6FAM\TTG AAT TGA TTT TGT ATA A IMGBNFQ\-3′. Cycling conditions were 95° C. for 5 mins, and 50 cycles of (95° C. for 30 s, 50° C. for 30 s and 72° C. for 30 s). PCR was conducted using a PCR buffer containing 16.6 mM (NH4)2SO4, 67 mM Tris (pH 8.8), 10 mM β-mercaptoethanol and magnesium chloride to yield a final magnesium concentration of 6.7 mM, 200 μM of each deoxynucleotide triphosphate (dNTP, MilliporeSigma) and 1 U of Platinum Taq polymerase (Thermo Fisher Scientific). The resulting DNA was bisulfite converted using the EZ DNA Methylation-Lightning™ Kit (ZYMO RESEARCH) according to the manufacturer's instructions and eluted into a final volume of 30 μL. DNA yields were quantified by LINE-1 quantification assay as described above. All synthetic control DNA was purchased from Integrated DNA Technologies (IDT) and used at concentrations according to the manufacturer's specifications.

### DREAMing Assay Validation

Assay validation on bulk was conducted with synthetic methylated DNA and bisulfite converted control genomic DNA. Approximately one genomic copy equivalent of methylated DNA and 100 or 500 copies of unmethylated control genomic DNA were mixed into a well of a 96-well microtiter plate. The presence of clearly distinguishable two melting peaks indicate the successful amplification of the methylated DNA among 100 or 500 copies of genomic background.

### Digital PCR and Digital Melt

Devices were placed on a flatbed heater (Biorad Proflex) with FC-40 oil between the glass and heater surface. PCR protocol was 95 C for 5 minutes, followed by 60 cycles of (95 C for 30 s, Ta for 30 s, and 72 C for 30 s), where Ta varied based on the target locus. Following PCR, devices were taken to the digital melt setup and secured on the heater via adhesive tape (3M). The heater ramped at 0.05 C/s, and images were taken with a full frame camera (Sony MILC) every second with 0.8 s exposure. A 480 nm LED array (Thorlabs) was used to illuminate the device, and fluorescence emission passed through a 570 nm bandpass filter (Edmunds Optics). Devices were imaged on a typhoon scanner (GE, 590 nm laser, 630 nm bandpass filter) to image the fluorescence of the control sequence probes.

### Image Processing and Data Analysis

Melt images were aligned after collection to correct for any thermally induced movement via an open-source automated program, Automated Image Registration (AIR). Fluorescence information was extracted from the images using Matlab as previously described. Fluorescence intensity values within each were averaged within 0.3° C. temperature intervals. Melt curves were generated by performing a low-pass and Savitzky-Golay filter on each well. The melt temperature of each amplicon was found by taking the negative derivative of the signal and identifying the location of the corresponding peak(s). Reference probe images were analyzed via the same mask generation, and fluorescence values within each well were averaged to calculate the signal. Positive wells for the reference molecule were identified via an arbitrary brightness threshold. The average melt temperature of the reference wells was used to set objective melt temperature thresholds which correlated the methylation density of the sample molecules. ROC curves were calculated using built-in functions in Matlab.

### Example 2: Use of Molecular Heterogeneity in DNA Methylation for Improving the Performance of Liquid Biopsy-Based Screening for Early-Stage (I & II) Non-Small Cell Lung Cancer (NSCLC)

Annual low-dose CT (LDCT) screening is currently recommended for adults aged 50 or older who are at high-risk of developing lung cancer. While this approach has resulted in improvements in survival, the false positive rate of lung nodules detection by LDCT remains over 95%, leading to unnecessary invasive follow-up procedures and further points to the need for new, complementary methods to improve diagnostics performance and reduce patient risk. DNA methylation biomarkers have demonstrated considerable potential as tumor-specific biomarkers for blood-based detection of early-stage NSCLC. Nonetheless, cell-free DNA (cfDNA) assessment techniques, such as methylation-specific PCR (MSP) or bisulfite sequencing, have limited sensitivity to assess epigenetic heterogeneity of rare epiallelic variants in a cost-effective manner. Here we reported a new platform, named REM-DREAMing (Ratiometric-Encoded Multiplex Discrimination of Rare EpiAlleles by Melt), which provides a simple, low-cost solution for multiplexed assessment of loci-specific DNA methylation heterogeneity at single molecule sensitivity. The microfluidic nanoarray contains four independent but identical 10,040 nanowell modules. Methylation biomarkers are differentiated by a ratiometric fluorescence scheme and the assessment of individual epiallele species of each locus are achieved through digitization in the nanoarray and precise high-resolution melt (HRM) analysis. In this study, we explore the potential utility of REM-DREAMing as a complementary assay for improving LDCT screening of NSCLC by testing a cohort of 48 clinical samples (28 cancer and 20 control samples) of low-volume liquid biopsy specimens from patients with CTscan indeterminant pulmonary nodules. A machine learning algorithm incorporating logistic regression models with leave-one-out cross validation was developed to identify a proper methylation density threshold of each biomarker in the panel. Evaluation of the receiver operation characteristic (ROC) curve yielded an area under the curve (AUC) of 0.97 (95% CI, 0.94-1) with 93% sensitivity at 95% specificity for the REM-DREAMing assay, compared with 93% sensitivity at 62% specificity achieved using a traditional, MSP-based approach. These results suggest that the assessment of intermolecular epigenetic heterogeneity can provide superior clinical performance for cfDNA methylation and noninvasive detection of early-stage NSCLC, in particular. Our results warrant further investigation in a larger sample cohort to validate its utility for improving routine screening of NSCLC in high-risk populations.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, kits, devices, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A method of determining methylation densities of target nucleic acids in a sample, the method comprising:
converting unmodified cytosine residues in nucleic acids from the sample to uracil residues to produce converted nucleic acids;
amplifying the target nucleic acids among the converted nucleic acids in multiplex reaction mixtures that comprise one or more sets of methylation-agnostic ratiometrically-encoded nucleic acid probes to produce detected target nucleic acid loci, wherein a given set of methylation-agnostic ratiometrically-encoded nucleic acid probes in a given reaction mixture comprises a sufficient number of sequence permutations to detect substantially all epialleles present at a given target nucleic acid locus and wherein the given set of methylation-agnostic ratiometrically-encoded nucleic acid probes comprises a predetermined ratio of differential labeling that produces a detectable signal that is sufficient to distinguish the detected epialleles present at the given target nucleic acid locus from epialleles detected at other target nucleic acid loci; and,
determining a methylation status of each epiallele detected at each detected target nucleic acid loci, thereby determining the methylation densities of the target nucleic acids in the sample.

2. The method of claim 1, wherein the target nucleic acids comprise at least about 10 different target nucleic acid loci, at least about 15 different target nucleic acid loci, at least about 25 different target nucleic acid loci, at least about 50 different target nucleic acid loci, at least about 75 different target nucleic acid loci, at least about 100 different target nucleic acid loci, or more different target nucleic acid loci.

3. The method of claim 1, wherein the target nucleic acid loci comprise cancer-specific methylation biomarkers.

4.-6. (canceled)

7. The method of claim 1, comprising classifying the methylation status of each epiallele detected at each detected target nucleic acid loci by determining melting temperatures of the epialleles, determining melting curve shapes of the epialleles, and/or thresholding corresponding methylation density histograms.

8. The method of claim 1, wherein the amplifying step comprises a methylation-independent amplification technique.

9. The method of claim 1, comprising diluting the reaction mixtures prior to amplifying the target nucleic acids among the converted nucleic acids such that the reaction mixtures each comprise at most about three epiallelic patterns.

10.-14. (canceled)

15. The method of claim 1, wherein the method produces absolute quantification over at least about five orders of magnitude.

16. (canceled)

17. The method of claim 1, further comprising detecting and identifying at least one disease state from the detected target nucleic acid loci and/or from the methylation densities of the target nucleic acids.

18. (canceled)

19. The method of claim 17, wherein the disease state comprises cancer and wherein the method further comprises identifying a tissue of origin of the cancer.

20.-22. (canceled)

23. The method of claim 1, wherein the reaction mixtures comprise one or more methylation-preferred nucleic acid primer pairs that amplify substantially all epiallelic fractions present at a given target nucleic acid locus irrespective of the methylation status of target nucleic acids comprising the given target nucleic acid locus in the sample.

24. The method of claim 1, wherein the differential labeling comprises at least a three-color ratiometric fluorescence encoding technique.

25. The method of claim 1, wherein the methylation status of each epiallele detected at each detected target nucleic acid loci is determined using at least a four-color fluorescence-decoding digital high resolution melt (dHRM) platform.

26. (canceled)

27. (canceled)

28. The method of claim 1, wherein the sets of methylation-agnostic ratiometrically-encoded nucleic acid probes comprise pan-cancer-detecting probes and cancer-identifying probes.

29.-31. (canceled)

32. A device, comprising a body structure that defines at least one inlet port, at least one fluidic channel, and at least one array of wells or droplets, which inlet port, fluidic channel, and array of wells or droplets fluidly communicate with one another,
wherein the wells or droplets each comprise a multiplex reaction mixture that comprises:
at most about one target nucleic acid, wherein unmodified cytosine residues in the target nucleic acid have been converted to uracil residues;
one or more methylation-preferred nucleic acid primer pairs and/or one or more methylation-agnostic nucleic acid primer pairs that are configured to amplify substantially all epiallelic fractions present at a given target nucleic acid locus irrespective of the methylation status of target nucleic acids comprising the given target nucleic acid locus;
one or more sets of methylation-agnostic ratiometrically-encoded nucleic acid probes, wherein a given set of methylation-agnostic ratiometrically-encoded nucleic acid probes is configured to produce differential detectable signal signatures for each different target nucleic acid loci in a sample when the target nucleic acid amplified and wherein the target nucleic acids in the sample comprise at least one of at least ten different target nucleic acid loci;
wherein the target nucleic acids in the multiplex reaction mixtures are configured to be amplified in the array of wells or droplets to produce detected target nucleic acid loci; and,
wherein epiallelic methylation densities at each detected target nucleic acid loci are configured to be determined in the array of wells or droplets.

33.-35. (canceled)

36. The device of claim 32, wherein a given set of methylation-agnostic ratiometrically-encoded nucleic acid probes is labeled with fluorescent labels that produces distinct three-color fluorescence ratios when amplifying difference target nucleic acid loci.

37. (canceled)

38. A kit comprising the device of claim 32.

39. A system, comprising a device having a body structure that defines at least one inlet port, at least one fluidic channel, and at least one array of wells or droplets, which inlet port, fluidic channel, and array of wells or droplets fluidly communicate with one another,
wherein the wells or droplets each comprise a multiplex reaction mixture that comprises: at most about one target nucleic acid, wherein unmodified cytosine residues in the target nucleic acid have been converted to uracil residues;
one or more methylation-preferred nucleic acid primer pairs and/or one or more methylation-agnostic nucleic acid primer pairs that are configured to amplify substantially all epiallelic fractions present at a given target nucleic acid locus irrespective of the methylation status of target nucleic acids comprising the given target nucleic acid locus;
one or more sets of methylation-agnostic ratiometrically-encoded nucleic acid probes, wherein a given set of methylation-agnostic ratiometrically-encoded nucleic acid probes is configured to produce differential detectable signal signatures for each different target nucleic acid loci in a sample when the target nucleic acid amplified and wherein the target nucleic acids in the sample comprise at least one of at least ten different target nucleic acid loci;
wherein the target nucleic acids in the multiplex reaction mixtures are configured to be amplified in the array of wells or droplets to produce detected target nucleic acid loci; and, wherein epiallelic methylation densities at each detected target nucleic acid loci are configured to be determined in the array of wells or droplets;
at least one thermal modulator that is configured to thermocycle the multiplex reaction mixtures to amplify the target nucleic acids and to perform melting temperature analyses of amplified target nucleic acids in the array of wells or droplets;
at least one detector that is capable of detecting detectable signals generated by the amplified target nucleic acids to produce the detected target nucleic acids and of detecting detectable signals corresponding to melting temperatures of the detected target nucleic acids to determine the epiallelic methylation densities at each detected target nucleic acid; and,
a controller operably connected to the thermal modulator and the detector, which controller is configured to modulate temperatures of the thermal modulator and to effect detection of the detectable signals from the detected target nucleic acids via the detector.

40. (canceled)

41. The system of claim 39, wherein the target nucleic acid loci comprise cancer-specific methylation biomarkers.

42. (canceled)

43. The system of claim 39, wherein a given set of methylation-agnostic ratiometrically-encoded nucleic acid probes is labeled with fluorescent labels that produces distinct three-color fluorescence ratios when amplifying difference target nucleic acid loci.

44. The system of claim 39, wherein the sets of methylation-agnostic ratiometrically-encoded nucleic acid probes comprise pan-cancer-detecting probes and cancer-identifying probes.
